(19) United States
(12) Patent Application Publication
(10) Pub. No.: US 2025/0003989 A1
(43) Pub. Date: Jan. 2, 2025

(54) METHODS FOR TAUOPATHY DIAGNOSIS AND TREATMENT

(71) Applicant: The Children`s Medical Center Corporation, Boston, MA (US)

(72) Inventors: Judith AJ Steen, Brighton, MA (US); Hanno Steen, Brighton, MA (US); Long Cheng, Brookline, MA (US); Pieter Beerepoot, Boston, MA (US)

(21) Appl. No.: 18/709,603

(22) PCT Filed: Nov. 15, 2022
(86) PCT No.: PCT/US2022/049920

§ 371 Date: May 13, 2024

Related U.S. Application Data
(60) Provisional application No. 63/279,544, filed on Nov. 15, 2021.

(51) Int. Cl. G01N 33/68; C07K 16/18; G01N 33/50

(52) U.S. Cl. CPC G01N 33/6896; C07K 16/18; G01N 33/502; G01N 2440/00; G01N 2500/10; G01N 2800/2821

(57) ABSTRACT

Provided herein are methods for treating a tauopathy in a subject in need thereof, the method comprising: identifying the subject having a tauopathy: administering to the subject an effective amount of a pharmaceutical composition comprising an agent that decreases the expression and/or activity of immunoproteasome (IP), decreases the expression and/or activity of an immunoproteasome-related protein whose level is upregulated in a subject having tauopathy, increases the expression and/or activity of an immuno-proteasome-related protein whose level is downregulated in a subject having tauopathy and increases the expression and/or activity of deubiquitinase while modulating inflammatory cytokine and cytokine receptor expression, thereby treating the tauopathy in the subject.

### CROSS-REFERENCE TO RELATED APPLICATIONS

This application claims priority to U.S. Provisional Patent Application No. 63/279,544, filed Nov. 15, 2021. The entire content of the foregoing application is incorporated herein by reference.

### FEDERALLY SPONSORED RESEARCH OR DEVELOPMENT

This invention was made with government support under grant number P30EY012196 awarded by the National Institutes of Health (NIH)/National Eye Institute (NEI). The government has certain rights in the invention.

### TECHNICAL FIELD

This disclosure relates to diagnosing and treating tauopathies, such as Alzheimer's disease (AD).

### BACKGROUND

The Tau pathology that is diagnostic of Alzheimer's Disease (AD) can spread from cell to cell, seeding aggregation of naïve Tau. The process that initiates aggregation has proved elusive, but is critical to the understanding of disease pathogenesis and therapeutic development.

A large and varied of group of neurodegenerative disorders are associated with neuropathological aggregates of the protein Tau, including the most common form of dementia, Alzheimer's Disease (AD). Monomeric Tau is a soluble cytosolic protein that can aggregate under pathological conditions to form highly organized, disease-specific filament structures (see, e.g., Falcon, B. et al. Novel tau filament fold in chronic traumatic encephalopathy encloses hydrophobic molecules. Nature 568, 420-423, doi: 10.1038/s41586-019-1026-5 (2019); Fitzpatrick, A. W. P. et al. Cryo-EM structures of tau filaments from Alzheimer's disease. Nature 547, 185-190, doi: 10.1038/nature23002 (2017); and Zhang, W. et al. Novel tau filament fold in corticobasal degeneration. Nature 580, 283-287, doi: 10.1038/s41586-020-2043-0 (2020)). Tau pathology spreads through functionally connected neurons in a manner consistent with an self-replicating proteinaceous particle, referred to as a prion (see, e.g., Prusiner, S. B. Novel proteinaceous infectious particles cause scrapie. Science 216, 136-144, doi: 10.1126/science.6801762 (1982)) or prion-like seed (see, e.g., Goedert, M., Clavaguera, F. & Tolnay, M. The propagation of prion-like protein inclusions in neurodegenerative diseases. Trends Neurosci 33, 317-325, doi: 10.1016/j.tins.2010.04.003 (2010)). However, there has been no comprehensive characterization of Tau cleavage sites in seed-competent Tau extracted from Alzheimer's patient brain tissue. Furthermore, the critical cause-and-effect question of whether cleavages promote Tau fibril formation or if Tau is cleaved after a fibril is formed has not been answered and is subject of active debate.

Thus, there is a need in the art for the diagnosis and treatment of tauopathies such as Alzheimer's Disease.

### SUMMARY

Provided herein are methods for treating a tauopathy in a subject in need thereof, the method comprising: (a) identifying the subject having a tauopathy: (b) administering to the subject an effective amount of a pharmaceutical composition comprising an agent that decreases the expression and/or activity of immunoproteasome (IP), thereby treating the tauopathy in the subject.

In some embodiments, the subject has Alzheimer's Disease (AD).

In some embodiments, the agent decreases the expression and/or activity of an immunoproteasome-related protein whose level is upregulated in a subject having tauopathy.

In some embodiments, the immunoproteasome-related protein is selected from the group consisting of PSMB 8, PSMB 9, PSMB 10, mitochondrial inner membrane protease, serine protease HTRA1, serine protease HTRA2, inactive Ufm 1-specific protease 1, calpain small subunit 1, thimet oligopeptidase, puromycin-sensitive aminopeptidase, signal peptidase complex subunit SEC11a, endoplasmic reticulum aminopeptidase 1, caspase 1, lysomal pro-X carboxypeptidase, peptidase inhibitor 16, prolyl endopeptidase, cytosol aminopeptidase, isoaspartyl peptidase, Xaa-pro dipeptidase, dipeptidyl aminopeptidase-like protein 16, isoaspartyl peptidase, cytosol aminopeptidase, probable aminopeptidase NPEPL1, prolyl endopeptidase, carboxypeptidase Q, puromycin-sensitive aminoprptidase, and dipeptidyl peptidase 3.

In some embodiments, the agent increases the expression and/or activity of an immunoproteasome-related protein whose level is downregulated in a subject having tauopathy.

In some embodiments, the agent increases the expression and/or activity of deubiquitinase.

In some embodiments, the agent decreases the expression and/or activity of a cytokine or a cytokine receptor.

In some embodiments, the cytokine is selected from the group consisting of IL-6, OSM (IL6 family), IL-1B, IL-15, IFN-γ, and TNF-α.

In some embodiments, the agent decreases the expression and/or activity of a transcription factor.

In some embodiments, the transcription factor is selected from the group consisting of STAT1/3, SMARCA4, and IRF2.

In some embodiments, the agent is a small molecule, a peptide, a stapled peptide, an siRNA, an antisense oligonucleotide (ASO), or an antibody.

In some embodiments, the agent reduces aggregation of tau peptides in the subject.

In some embodiments, the agent reduces seeding competence of tau peptides in the subject.

In some embodiments, the agent reduces fibrillization of tau peptides in the subject.

Also provided herein are methods for screening a therapeutic agent for treating a tauopathy, the method comprising: (a) providing a plurality of cells that express tau peptides; (b) subjecting the plurality of cells to one or more stress signals, wherein the stress signals induce the aggregation of tau peptides: (c) contacting the plurality of cells from (b) with a candidate therapeutic agent: (d) comparing the levels of tau peptide aggregation before and after the contacting of the candidate therapeutic agent: (e) selecting the therapeutic agent if the aggregation of tau peptides is decreased. thereby screening the therapeutic agent.

In some embodiments, the method further comprises measuring aggregation of tau peptide prior to (d).

In some embodiments, the method further comprises identifying fragments of tau peptides that comprises one or more cleavage sites.

In some embodiments, the fragments of tau peptide is identified using FLEXITau.

In some embodiments, the therapeutic agent targets one or more cleavage sites on tau peptide.

In some embodiments, the therapeutic agent is an antibody.

In some embodiments, the therapeutic agents targets one or more of post-translational modifications (PTMs) on tau peptide.

In some embodiments, the tauopathy is Alzheimer's Disease (AD).

In some embodiments, the therapeutic agent decreases the expression and/or activity of an immunoproteasome-related protein whose level is upregulated in a subject having tauopathy.

In some embodiments, the therapeutic agent increases the expression and/or activity of an immunoproteasome-related protein whose level is downregulated in a subject having tauopathy.

In some embodiments, the therapeutic agent decreases the expression and/or activity of a cytokine or a cytokine receptor.

In some embodiments, the therapeutic agent decreases the expression and/or activity of a transcription factor.

In some embodiments, the therapeutic agent is a small molecule, a peptide, a stapled peptide, an siRNA, an antisense oligonucleotide (ASO), or an antibody.

In some embodiments, the therapeutic agent reduces aggregation of tau peptides in the subject.

In some embodiments, the therapeutic agent reduces seeding competence of tau peptides in the subject.

In some embodiments, the therapeutic agent reduces fibrillization of tau peptides in the subject.

Unless otherwise defined, all technical and scientific terms used herein have the same meaning as commonly understood by one of ordinary skill in the art to which this invention belongs. Methods and materials are described herein for use in the present invention: other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-1E depict the cleavages in pathological Tau isolated from human brain tissue with Alzheimer's disease cluster around the aggregation prone core domain. Cleavages were mapped in different biochemical preparations at different stages of aggregation/solubility from soluble to insoluble fibrillar Tau, and in sarkosyl soluble (SS) or sarkosyl insoluble (SI) preparations from both CTRL (N=27) and AD (N=24). Using FLEXITau, the extent of modification on Tau peptides was determined (overlay on cleavage map) showing that peptides in the region between Q244 and K395 have low levels of modification (inner boundary, dark blue) whereas peptides outside these regions were highly modified (outer boundary includes cleavages in neighboring peptides. Light blue). FIG. 1A shows the SS cleavage map; FIG. 1B shows the population frequency of cleavages in sarkosyl soluble fraction; FIG. 1C shows the SI cleavage map; FIG. 1D shows patient frequency of semitryptic cleavages in CTRL vs AD insoluble Tau, which indicates that semitryptic peptides are present at high frequency only in AD but not in CTRL; and putative protofilament core peptides generated by these cleavages are outlined in FIG. 1E. Consistent cleavage hotspots (N-terminal Q244, 1297/S304/S305 and C-terminal N368, and Y394) were observed in seed-competent vs inert Tau preparations clustering around the enriched microtubule binding domain region.

FIG. 2A-2F depict the proteomics of SI fraction from post-mortem AD tissue, which shows upregulation of antigen presentation pathway and associated proteolytic enzymes. FIG. 2A is a volcano plot showing differential protein expression in AD vs CTRL. Significantly changed proteins (highlighted in grey) were then cross-referenced with the MEROPS peptidase database to identify proteases that were upregulated (blue) or downregulated (red). Differentially expressed proteases were validated for reported protease (black text)/lack of protease activity (grey text). Color coding represents log 2 fold change. Four out of five upregulated proteases are positively regulated by interferon gamma and/or TNF alpha signaling. FIG. 2B shows the correlations between upregulated proteases and intensity of cleavage site-specific peptides. FIG. 2C shows the overrepresented reactome pathways in upregulated SI proteins. FIG. 2D shows the upstream cytokine and transcription factor regulators of changes in the SI protein abundance identified using Ingenuity Pathway Analysis, which indicates upregulation of inflammatory cytokines including IFN/TNF. FIGS. 2E-2F show the multiplexed immunostaining of frontal cortex tissue. Arrows-neurofibrillary tangles with colocalized phospho Tau and PSMB8 immunoreactivity.

FIGS. 3A-3F depict that recombinant Tau peptides defined by cleavages in post-mortem AD tissue can fibrillize in vitro and resultant fibrils can seed aggregation in HEK293T cells stably expressing Tau-K18. FIG. 3A depicts putative core cleavage fragments mapped to the cryo-EM structure of the protofilament, showing that cleavage at N368 falls within the core structure. FIG. 3B depicts that Tht confirmed these fragment peptides can primarily form fibrils and resulted fibril can seed endogenous Tau-K18 to aggregate in HEK293T cells. Aggregation shown as maximum Tht after 15 hours. FIG. 3C shows that preformed fibrils of Tau fragments trigger aggregation in HEK293T cells stably expressing Tau-K18. Seeding assays show varying degrees of efficacy for each fragment shown as % of successfully seeded cells. It is noted, peptides cleaved less at C-terminus (b type) show higher potency of fibrillization and seeding over the other (a type). FIG. 3D is a schematic illustration of in vitro seeding tests showing 1^{st} generation fibrils were formed by addition of heparin to cause aggregation (left column). FIG. 3E shows the second generation seeding of naïve Tau peptide with 2b or Tau 187 seeds. Tht values from each panel in FIG. 3D were normalized by the mass of cofactor and protein used. Stars indicates significance from Mann-Whitney-Wilcoxon test. FIG. 3F is a schematic of 2^{nd} generation fibrils formed with a dilution of 1^{st} generation fibrils by fresh protein. Fibrils were confirmed by Tht and TEM (right column).

FIGS. 4A-4E depict that interferon γ/TNFα treatment leads to Tau cleavage, and accumulation of hyperphosphorylated Tau in insoluble aggregates in HEK293T cells stably expressing full-length Tau (0N4R-P301S). FIG. 4A shows the immunostaining of HEK293T cells stably expressing C-terminal EYFP-fused full-length Tau (0N4R-P301S) after 3, 5, or 8 days of treatment with vehicle or Interferon γ/TNFα (200 ng/ml for each). Green shows the signal of EYFP which is originally fused to C-terminus of expressed full-length Tau (0N4R-P301S); red shows the signal of immunostaining against anti-Tau RD4 antibody recognizing epitope located at amino acid residues 275-291 of human R4 Tau; blue shows DAPI staining. Scale bar, 50 μm. Arrows highlighted the Tau accumulation induced by cytokines treatment, which is anti-Tau RD4 positive but EYFP negative. FIG. 4B shows the enriched pathways in day 8 cell lysate proteomics. FIG. 4C shows the fold change of IFN/TNF treated cells compared to control lproteasome subunits in label free proteomics data of day 8 cell lysate. Hyperphosphorylation of Tau epitopes after cytokine treatment. FIG. 4D shows the immunoblot of treated and untreated day 8 cell lysates, probed with YFP antibody (green) and Tau RD4 antibody (red). FIG. 4E shows the summary of detected semitryptic peptides in different Tau preparations.

FIGS. 5A-5C depict the knock-down of immunoproteasome subunits reduces Interferon γ/TNFα-induced Tau aggregates in HEK293T cells stably expressing full-length Tau (0N4R-P301S). FIG. 5A shows the immunoblot of cell lysates of HEK293T cells stably expressing C-terminal EYFP-fused full-length Tau (0N4R-P301S) transfected with 200 nM control, PSMB10 or PSMB9 SiRNA, respectively, a day prior to 3 or 6 days of treatment with vehicle or Interferon γ/TNFα (100 ng/ml for each). The primary antibodies probing PSM10, 9, 8 were used. FIG. 5B shows the immunostaining of cells in FIG. 5A, which were transfected with above SiRNA and 6 days treatment of vehicle or Interferon γ/TNFα (100 ng/ml for each). Green shows the signal of EYFP; red shows the signal of immunostaining against anti-Tau RD4 antibody; blue shows DAPI staining. Scale bar, 50 μm. FIG. 5C shows the cell toxicity measured by LDH activity in cell culture medium with 6 days of treatment with vehicle or Interferon γ/TNFα at various doses.

FIGS. 6A-6D. Tau cleavages identified in seeding competent and seeding incompetent Tau preparations from human postmortem tissue. FIG. 6A shows the cleavage of LMW Tau. FIG. 6B shows the cleavages of HWM Tau. FIG. 6C shows the cleavages of MC1-purified Tau. FIG. 6D shows the cleavages of SI Tau.

FIG. 7 depicts the abundance of FLEXITau relative unmodified peptide.

FIGS. 8A-8B depicts the differential protein abundance in AD vs NDC sarkosyl soluble fraction. FIG. 8A is a volcano plot showing differential protein abundance. The downregulated proteins highlighted in red and upregulated proteins highlighted in blue in FIG. 8B.

FIGS. 9A-9B depict the in vitro degradation of 0N4R recombinant Tau by the immunoproteasome. FIG. 9A shows the silverstained SDS-PAGE gel of Tau incubated in the presence or absence of immunoproteasome (n=3). FIG. 9B shows the IP cleavage sites in recombinant 0N4R Tau, visualized on the sequence of 2N4R Tau (n=3). Correlations were calculated. FDR cut off was set by calculating correlations for a same-sized population of randomly chosen semitryptic peptides belonging to other protein groups.

FIG. 10 is a panel of original scans of the immunoblots in FIG. 5A. It is noted that each experiment was shown in experimental duplicate.

FIG. 11 is a schematic illustration of an example screening assay described herein.

### DETAILED DESCRIPTION

Provided herein are method for diagnosing and treating tauopathies, e.g., Alzheimer's Disease (AD). Particularly, the methods include inhibiting immunoproteasomes (IPs) or an immunoproteasome-related protein. Also provided herein are methods for screening a therapeutic agent for treating tauopathies, e.g., Alzheimer's Disease (AD).

The present disclosure is based, at least partially, on the discovery that neuronal immunoproteasome (IP) induction links inflammatory signaling to pathological Tau aggregation in Alzheimer's Disease. The proteolytic cleavage of Tau is associated with stress and inflammation pathways that result in immunoproteosome (IP) expression in neurons in the AD brain.

Accordingly, in one aspect, provided herein are methods for treating a tauopathy in a subject in need thereof, the method comprising: (a) identifying the subject having a tauopathy: (b) administering to the subject an effective amount of a pharmaceutical composition comprising an agent that decreases the expression and/or activity of immunoproteasome (IP), thereby treating the tauopathy in the subject.

### Tauopathies

Human Tau is encoded on chromosome 17q21 (see, e.g., Neve R L et al., Brain Res. 1986 December; 387 (3); 271-80). The protein occurs mainly in the axons of the central nerve system (CNS) and consists largely of six isoforms generated by alternative splicing (see, e.g., Goedert M et al., EMBO J. 1989 February; 8 (2); 393-9). They differ by the presence or absence of two near-amino-terminal inserts of 29 residues each, encoded by exons 2 and 3, and by one of the repeats (R2, 31 residues) in the carboxy-terminal half. A representative sequence of human tau 2N4R isoform is shown below:

Amino Acid Sequence of 2N4R Tau Protein
>sp|P10636-8|TAU_HUMAN Isoform Tau-F of Micro-
tubule-associated protein tau
OS = Homo sapiens OX = 9606 GN = MAPT
SEQ ID NO: 1
MAEPRQEFEVMEDHAGTYGLGDRKDQGGYTMHQDQEGDTDAGLKESPLQT
PTEDGSEEPGSETSDAKSTPTAEDVTAPLVDEGAPGKQAAAQPHTEIPEG
TTAEEAGIGDTPSLEDEAAGHVTQARMVSKSKDGTGSDDKKAKGADGKTK
IATPRGAAPPGQKGQANATRIPAKTPPAPKTPPSSGEPPKSGDRSGYSSP
GSPGTPGSRSRTPSLPTPPTREPKKVAVVRTPPKSPSSAKSRLQTAPVPM
PDLKNVKSKIGSTENLKHQPGGGKVQIINKKLDLSNVQSKCGSKDNIKHV
PGGGSVQIVYKPVDLSKVTSKCGSLGNIHHKPGGGQVEVKSEKLDFKDRV
QSKIGSLDNITHVPGGGNKKIETHKLTFRENAKAKTDHGAEIVYKSPVVS
GDTSPRHLSNVSSTGSIDMVDSPQLATLADEVSASLAKQGL

Tauopathies represent a large group of proteopathies featuring aggregates of an altered form of the microtubule associated protein tau. The term “tauopathy” refers to tau-related disorders or conditions, e.g., Alzheimer's Disease (AD), Progressive Supranuclear Palsy (PSP), Corticobasal Degeneration (CBD), Pick's Disease (PiD), Argyrophilic grain disease (AGD), Frontotemporal dementia and Parkinsonism associated with chromosome 17 (FTDP-17), Parkinson's disease, stroke, traumatic brain injury, mild cognitive impairment and the like.

Alzheimer's disease (AD) is a kind of tauopathy. It is a chronic neurodegenerative disease. The most common early symptom is difficulty in remembering recent events (short-term memory loss). As the disease advances, symptoms can include problems with language, disorientation (including easily getting lost), mood swings, loss of motivation, not managing self-care, and behavioral issues.

Braak staging is often used to classify the degree of pathology in Alzheimer's disease. The first two stages are characterized by an either mild or severe alteration of the transentorhinal layer Pre-alpha (transentorhinal stages I-II). The two forms of limbic stages (stages III-IV) are marked by a conspicuous affection of layer Pre-alpha in both transentorhinal region and proper entorhinal cortex. In addition, there is mild involvement of the first Ammon's horn sector. The hallmark of the two isocortical stages (stages V-VI) is the destruction of virtually all isocortical association areas. A detailed description of Braak staging can be found e.g., in Braak et al., “Neuropathological stageing of Alzheimer-related changes.” Acta neuropathologica 82.4 (1991); 239-259, which is incorporated herein by reference in its entirety.

The pathway leading from soluble and monomeric to hyperphosphorylated, insoluble and filamentous tau protein is at the center of tauopathies. Usually, the first tau aggregates form in a few nerve cells in discrete brain areas. These become self propagating and spread to distant brain regions in a prion-like manner. In a clinical setting, the clinical syndromic diagnosis is often determined by the patient's symptoms and deficits, while the pathological diagnosis is defined by characteristic types and distribution of the tau inclusions and of neuron loss. In some embodiments, the subject has Alzheimer's Disease (AD).

### Subjects

The terms “subject” and “patient” are used interchangeably throughout the specification and describe an animal, human or non-human, to whom treatment according to the methods of the present invention is provided. Human patients can be adult humans or juvenile humans. In some embodiments, humans can have an age of above 10, 20, 30, 35, 40, 45, 50, 55, 60, 65, 70, 75, 80, 85, or 90 years old. In some embodiments, the subject is a mammal. In some embodiments, the term “subject”, as used herein, refers to a human (e.g., a man, a woman, or a child).

The subject can be symptomatic (e.g., the subject presents symptoms associated with tauopathies (e.g., AD, AGD, CBD, PiD, PSP), such as, for example changes in personality, behavior, sleep patterns, and executive function, memory loss, confusion, inability to learn new things, difficulty carrying out multistep tasks, problems coping with new situations, hallucinations, delusions, and paranoia, impulsive behavior, inability to communicate, weight loss, seizures, skin infections, difficulty swallowing, groaning, moaning, grunting, increased sleeping, lack of control of bowel and bladder, disorders of word finding, disorders of reading and writing, disorientation, supranuclear palsy, a wide-eyed appearance, difficulty in swallowing, unwarranted anxiety, irrational fears, oniomania, impaired regulation of social conduct (e.g., breaches of etiquette, vulgar language, tactlessness, disinhibition, misperception), passivity, low motivation (aboulia), inertia, over-activity, pacing and wandering, etc. The subject can be asymptomatic (e.g., the subject does not present symptoms associated with a tauopathy, or the symptoms have not been recognized).

In addition to humans, subjects include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

In some embodiments, the subject is a human subject.

### Sample Collection and Preparation

Samples for use in the methods described herein include various types of samples from a subject.

In some embodiments, the sample is a “biologic sample”. As used herein, the term “biological sample” or “sample” refers to a sample obtained or derived from a subject. By way of example, the sample may be selected from the group consisting of body fluids, blood, whole blood, plasma, serum, mucus secretions, urine or saliva. In some embodiments the sample is, or comprises a blood sample. The preferred biological source for detection of the biomarkers is a blood sample, a serum sample or a plasma sample. In some embodiments, the sample is cerebrospinal fluid (CSF) or a brain tissue.

As used herein, “obtain” or “obtaining” can be any means whereby one comes into possession of the sample by “direct” or “indirect” means. Directly obtaining a sample means performing a process (e.g., performing a physical method such as extraction) to obtain the sample. Indirectly obtaining a sample refers to receiving the sample from another party or source (e.g., a third party laboratory that directly acquired the sample). Directly obtaining a sample includes performing a process that includes a physical change in a physical substance, e.g., a starting material, such as a blood, e.g., blood that was previously isolated from a patient. Thus, obtain is used to mean collection and/or removal of the sample from the subject. Furthermore, “obtain” is also used to mean where one receives the sample from another who was in possession of the sample previously.

In some embodiments, a reference sample is obtained from at least one individual not suffering from a tauopathy. In some other embodiments, the reference sample is obtained from at least one individual previously diagnosed as having a tauopathy (e.g., AD, AGD, CBD, PiD, PSP). In some embodiments, the reference sample comprises a predetermined, statistically significant reference analyte levels.

In some embodiments, the sample is collected from the brain of a subject, e.g., brain tissue. In some embodiments, the sample is collected from cerebrospinal fluid or plasma.

In some embodiments, the sample is collected from a biopsy. A biopsy is a sample of tissue taken from the body of a living subject. A biopsy sometimes also refers to the medical procedure that removes tissue from a living subject. In some embodiments, the sample can be collected through a punch biopsy. A punch biopsy is done with a circular blade ranging in size from 1 mm to 8 mm. In some embodiments, the sample can be collected from fine-needle aspiration biopsy (FNAB or FNA). Fine-needle aspiration biopsy is a procedure used to investigate superficial (just under the skin) lumps or masses. In some embodiments, a thin, hollow needle is inserted into the body to collect samples.

In some embodiments, the sample is from a live subject. For example, the sample can be collected from a subject during a medical procedure, e.g., a surgery.

In some embodiments, samples are collected from post-mortem specimens, e.g., human post-mortem brain specimens.

In some embodiments, brain tissue can be obtained from Brodmann area 39 (BA39) angular gyrus brain blocks.

In some embodiments, biopsy samples are homogenized and clarified by centrifugation. Supernatants containing tau proteins are pooled and used as a crude tau fraction (unfractionated homogenate).

In some embodiments, samples are collected from cultured cells, e.g., from E. coli or sf9 cells. In some embodiments, samples are collected from the brain tissue of model animals.

### Immunoproteasome (IP)

The methods provided herein include targeting immunoproteasome (IP) for the treatment of tauopathies. The immunoproteasome is a highly efficient proteolytic machinery derived from the constitutive proteasome and is abundantly expressed in immune cells. The immunoproteasome plays a critical role in the immune system because it degrades intracellular proteins, for example, those of viral origin, into small proteins (see, e.g., Kimura et al., Journal of Immunology Research Volume 2015, Article ID 541984). Immunoproteasomes contain replacements for the three catalytic subunits of standard proteasomes. In most cells, oxidative stress and proinflammatory cytokines are stimuli that lead to elevated production of immunoproteasomes. Immune system cells, especially antigen-presenting cells, express a higher basal level of immunoproteasomes. A well-described function of immunoprotea-somes is to generate peptides with a hydrophobic C terminus that can be processed to fit in the groove of MHC class I molecules. This display of peptides on the cell surface allows surveillance by CD8 T cells of the adaptive immune system for pathogen-infected cells (see, e.g., Ferrington et al., Prog Mol Biol Transl Sci. 2012; 109: 75-112).

In some embodiments, the immunoproteasome-related protein of the method described herein is selected from PSMB 8, PSMB 9, PSMB 10, mitochondrial inner membrane protease, serine protease HTRA1, serine protease HTRA2, inactive Ufm1-specific protease 1, calpain small subunit 1, thimet oligopeptidase, puromycin-sensitive aminopeptidase, signal peptidase complex subunit SEC11a, endoplasmic reticulum aminopeptidase 1, caspase 1, lysomal pro-X carboxypeptidase, peptidase inhibitor 16, prolyl endopeptidase, cytosol aminopeptidase, isoaspartyl peptidase, Xaa-pro dipeptidase, dipeptidyl aminopeptidase-like protein 16, isoaspartyl peptidase, cytosol aminopeptidase, probable aminopeptidase NPEPL1, prolyl endopeptidase, carboxypeptidase Q, puromycin-sensitive aminoprptidase, and dipeptidyl peptidase 3.

In some embodiments, an immunoproteasome-related protein is a proteolytic enzyme. Examples of proteolytic enzymes are shown in Table 1 below.

TABLE 1
Examples of proteolytic enzymes

| Global | |
|---|---|
| Protein Name | UniP |
| Mitochondrial inner membrane protease | ATP23 |
| Serine protease HTRA1 | HTRA1 |
| Serine protease HTRA2 | HTRA2 |
| Inactive Ufm 1-specific protease 1 | UFSP1 |
| Calpain small subunit 1 | CPNS1 |
| Thimet oligopeptidase | THOP1 |
| Puromycin-sensitive aminopeptidase | PSA |
| Signal peptidase complex subunit SEC11a | SEC11A |
| Endoplasmic reticulum aminopeptidase 1 | ERAP1 |
| Caspase 1 | CASP1 |
| Lysosomal Pro-X carboxypeptidase | PCP |
| Peptidase inhibitor 16 | PI16 |
| Soluble/Insoluble | ANOVA |
| Soluble | |
| Prolyl endopeptidase | PPCE |
| Cytosol aminopeptidase | AMPL |
| Isoaspartyl peptidase | ASGL1 |
| Xaa-Pro dipeptidase | PEPD |
| Insoluble | |
| Dipeptidyl aminopeptidase-like protein 6 | DPP6 |
| Isoaspartyl peptidase | ASGL1 |
| Cytosol aminopeptidase | AMPL |
| Probable aminopeptidase NPEPL1 | PEPL1 |
| Prolyl endopeptidase | PPCE |
| Carboxypeptidase Q | CBPM |
| Puromycin-sensitive aminopeptidase | PSA |
| Dipeptidyl peptidase 3 | DPP3 |

The agent used in the methods described herein can be any suitable agent known in the art. In some embodiments, the agent is a small molecule, a peptide, a stapled peptide, an siRNA, an antisense oligonucleotide (ASO), or an antibody.

In some embodiments, the agent targets (e.g., increase or decrease the expression and/or activity of) ATP23, HTRA1, HTRA2, UFSP1, CPNS1, THOP1, PSA, SEC11A, ERAP1, CASP1, PCP, PI16, PPCE, AMPL, ASGL1, PEPD, DPP6, ASGL1, AMPL, PEPL1, PPCE, CBPM, PSA, or DPP3.

In some embodiments, the agent increases the expression and/or activity of an immunoproteasome-related protein whose level is downregulated in a subject having tauopathy. In some embodiments, the expression and/or activity of the immunoproteasome-related protein is increased by about 5%, 10%, 15%, 20%, 25%, 30%, 35%, 40%, 45%, 50%, 55%, 60%, 65%, 70%, 75%, 80%, 85%, 90%, 95% or more. In some embodiments, the increase of expression and/or activity of an immunoproteasome-related protein is measured by comparison with a reference expression and/or activity. In some embodiments, the reference expression and/or activity is an expression and/or activity of the immunoproteasome-related protein in a subject without or before the administration of the agent.

In some embodiments, the agent decreases the expression and/or activity of an immunoproteasome-related protein whose level is upregulated in a subject having tauopathy. In some embodiments, the expression and/or activity of the immunoproteasome-related protein is decreased by about 5%, 10%, 15%, 20%, 25%, 30%, 35%, 40%, 45%, 50%, 55%, 60%, 65%, 70%, 75%, 80%, 85%, 90%, 95% or more. In some embodiments, the decrease of expression and/or activity of an immunoproteasome-related protein is measured by comparison with a reference expression and/or activity. In some embodiments, the reference expression and/or activity is an expression and/or activity of the immunoproteasome-related protein in a subject without or before the administration of the agent.

In some embodiments, the agent increases the expression and/or activity of deubiquitinase.

In some embodiments, the agent reduces aggregation of tau peptides in the subject. In some embodiments, the aggregation of tau peptides is reduced by about 5%, 10%, 15%, 20%, 25%, 30%, 35%, 40%, 45%, 50%, 55%, 60%, 65%, 70%, 75%, 80%, 85%, 90%, 95% or more. In some embodiments, the reduction of aggregation of tau peptides is measured by comparison with a reference aggregation level. In some embodiments, the reference aggregation level is an aggregation level of tau peptides in a subject without or before the administration of the agent.

In some embodiments, the agent reduces seeding competence of tau peptides in the subject. In some embodiments, the seeding competence of tau peptides is reduced by about 5%, 10%, 15%, 20%, 25%, 30%, 35%, 40%, 45%, 50%, 55%, 60%, 65%, 70%, 75%, 80%, 85%, 90%, 95% or more. In some embodiments, the reduction of seeding competence of tau peptides is measured by comparison with a reference level. In some embodiments, the reference level is the seeding competence of tau peptides in a subject without or before the administration of the agent.

In some embodiments, the agent reduces fibrillization of tau peptides in the subject. In some embodiments, the fibrillization of tau peptides is reduced by about 5%, 10%, 15%, 20%, 25%, 30%, 35%, 40%, 45%, 50%, 55%, 60%, 65%, 70%, 75%, 80%, 85%, 90%, 95% or more. In some embodiments, the reduction of fibrillization of tau peptides is measured by comparison with a reference level. In some embodiments, the reference level is the fibrillization of tau peptides in a subject without or before the administration of the agent.

### Administration of the Agents

In some embodiments, the methods include administering agents, e.g., inhibitors of immunoproteasome-related proteins, to the subject. Any suitable administration methods known in the art can be used in the methods described herein.

In some embodiments, the methods described herein include the use of pharmaceutical compositions comprising the agent for the inhibition of the expression and/or activity of immunoproteasome (IP).

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art (see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active composition in the required amount in an appropriate solvent with one or a combination of ingredients enumerated above, as required, followed by filtered sterilization. Generally, dispersions are prepared by incorporating the active composition into a sterile vehicle, which contains a basic dispersion medium and the required other ingredients from those enumerated above.

The pharmaceutical compositions can be included in a container, pack, or dispenser together with instructions for administration.

### Dosage

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of an active agent (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Screening Methods

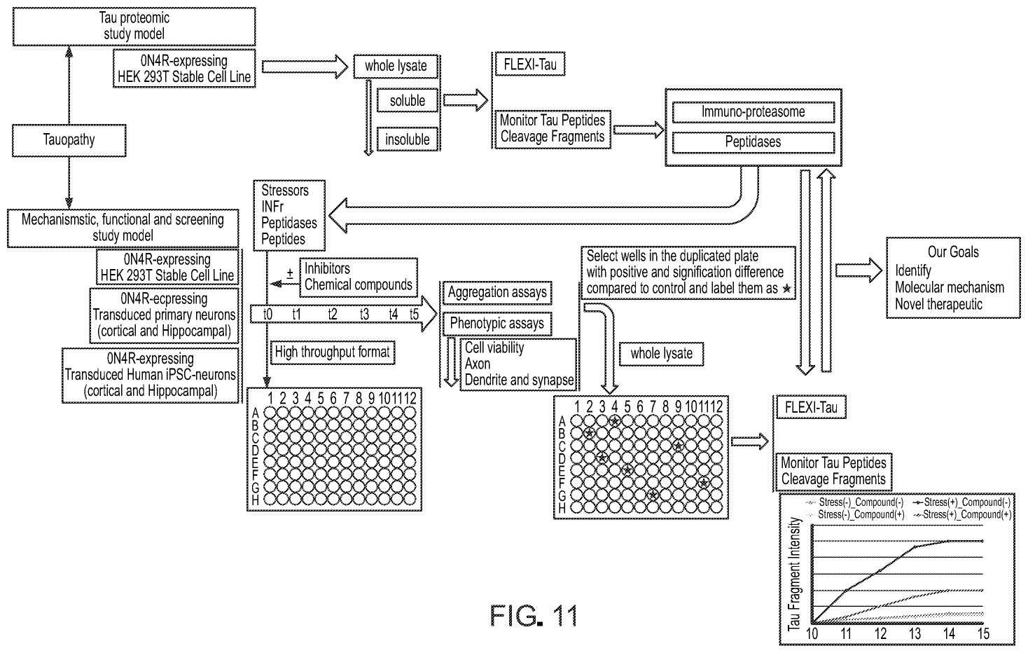

In one aspect, provided herein are methods for screening a therapeutic agent for treating a tauopathy, the method comprising: (a) providing a plurality of cells that express tau peptides: (b) subjecting the plurality of cells to one or more stress signals, wherein the stress signals induce the aggregation of tau peptides: (c) contacting the plurality of cells from (b) with a candidate therapeutic agent: (d) comparing the levels of tau peptide aggregation before and after the contacting of the candidate therapeutic agent: (e) selecting the therapeutic agent if the aggregation of tau peptides is decreased. thereby screening the therapeutic agent. In some embodiments, some of these steps are shown in detail in FIG. 11.

In some embodiments, the methods for screening include screening with imaging analysis in a cell system and then with FLEXIQuant to see the reduction of aggregates and ubiquitinated tau with the most effective inhibitors. Specifically for screening of inhibitors of IP-mediated tau cleavage, tau labeled with an N-terminal fluorophore, N-terminal and C-terminal FRET pair, or other reporter could be used. Cleavage at IP specific sites would be detected by change in fluorescence, reduction in FRET signal, or conversion of an enzymatic substrate.

In some embodiments, the methods described herein further include measuring aggregation of tau peptide prior to comparing the levels of tau peptide aggregation before and after the contacting of the candidate therapeutic agent.

In some embodiments, the methods described herein further include identifying fragments of tau peptides that comprises one or more cleavage sites. Examples of cleavage sites are described herein.

In some embodiments, the fragments of tau peptide is identified using FLEXITau (see, e.g., Mair W et al., Anal Chem. 2016 Apr. 5; 88 (7); 3704-14; and PCT Publication WO 2022/104136A2).

In some embodiments, the therapeutic agent targets one or more cleavage sites on tau peptide. In some embodiments, the therapeutic agent is an antibody.

Screening can be performed in a high through-put and quantitative system using biosensor cells expressing genetically engineered Tau with fluorescent reporters on the N or C terminus, whereby the changes in Tau cleavage and aggregation are measured by analyzing the morphology, intensity, location and FRET vale of fluorescent reporters as well as using FLEXITau and mass spectrometry workflows.

The test compounds can be, e.g., natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio. 1:60-6 (1997)). In addition, a number of small molecule libraries are commercially available. A number of suitable small molecule test compounds are listed in U.S. Pat. No. 6,503,713, incorporated herein by reference in its entirety.

Libraries screened using the methods of the present invention can comprise a variety of types of test compounds. A given library can comprise a set of structurally related or unrelated test compounds. In some embodiments, the test compounds are peptide or peptidomimetic molecules. In some embodiments, the test compounds are nucleic acids.

In some embodiments, the test compounds and libraries thereof can be obtained by systematically altering the structure of a first test compound, e.g., a first test compound that is structurally similar to a known natural binding partner of the target polypeptide, or a first small molecule identified as capable of binding the target polypeptide, e.g., using methods known in the art or the methods described herein, and correlating that structure to a resulting biological activity, e.g., a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a small molecule compound or compounds. For example, in one embodiment, a general library of small molecules is screened, e.g., using the methods described herein.

In some embodiments, a test compound is applied to a test sample, e.g., a protein sample, a cell or living tissue or organ, and one or more effects of the test compound is evaluated. In a cultured or primary cell for example, the ability of the test compound to inhibit the PTM of interest or promote the PTM of interest is determined.

In some embodiments, the test sample is, or is derived from (e.g., a sample taken from) an in vivo model of a disorder as described herein. For example, an animal model, e.g., a rodent such as a rat, can be used.

Methods for evaluating each of these effects are known in the art. For example, ability to modulate expression of a protein can be evaluated at the gene or protein level, e.g., using quantitative PCR or immunoassay methods. In some embodiments, high throughput methods, e.g., protein or gene chips as are known in the art (see, e.g., Ch. 12, Genomics, in Griffiths et al., Eds. Modern genetic Analysis, 1999, W. H. Freeman and Company: Ekins and Chu, Trends in Biotechnology, 1999, 17:217-218; MacBeath and Schreiber, Science 2000, 289 (5485); 1760-1763: Simpson, Proteins and Proteomics: A Laboratory Manual, Cold Spring Harbor Laboratory Press: 2002; Hardiman, Microarrays Methods and Applications: Nuts & Bolts, DNA Press, 2003), can be used to detect an effect on PTMs. Ability to modulate PTMs can be evaluated, e.g., using methods as described in this disclosure.

A test compound that has been screened by a method described herein and determined to inhibit PTMs of interest, or inhibit tau protein aggregation, or promote the PTM of interest can be considered a candidate compound. A candidate compound that has been screened, e.g., in an in vivo model of a disorder, e.g., AD, PSP, CBD, PID, AGD, and determined to have a desirable effect on the disorder, e.g., on one or more symptoms of the disorder, can be considered a candidate therapeutic agent. Candidate therapeutic agents, once screened in a clinical setting, are therapeutic agents. Candidate compounds, candidate therapeutic agents, and therapeutic agents can be optionally optimized and/or derivatized, and formulated with physiologically acceptable excipients to form pharmaceutical compositions.

Thus, test compounds identified as “hits” (e.g., test compounds that have the ability to inhibit certain PTMs, promote certain PTM, or inhibit tau protein aggregations) in a first screen can be selected and systematically altered, e.g., using rational design, to optimize binding affinity, avidity, specificity, or other parameter. Such optimization can also be screened for using the methods described herein. Thus, in one embodiment, the invention includes screening a first library of compounds using a method known in the art and/or described herein, identifying one or more hits in that library, subjecting those hits to systematic structural alteration to create a second library of compounds structurally related to the hit, and screening the second library using the methods described herein.

Test compounds identified as hits can be considered candidate therapeutic compounds, useful in treating tauopathies, e.g., AD, AGD, CBD, PiD, PSP, or symptoms associated with tauopathies. A variety of techniques useful for determining the structures of “hits” can be used in the methods described herein, e.g., NMR, mass spectrometry, gas chromatography equipped with electron capture detectors, fluorescence and absorption spectroscopy. Thus, the invention also includes compounds identified as “hits” by the methods described herein, and methods for their administration and use in the treatment, prevention, or delay of development or progression of a disorder described herein.

Test compounds identified as candidate therapeutic compounds can be further screened by administration to an animal model of a tauopathy (e.g., AD, AGD, CBD, PiD, PSP), as described herein. The animal can be monitored for a change in the disorder, e.g., for an improvement in a parameter of the disorder, e.g., a parameter related to clinical outcome. In some embodiments, the parameter is memory, and an improvement would be an increase in short-term memory.

### Post Translational Modifications (PTMs)

In some embodiments, the therapeutic agents targets one or more of post-translational modifications (PTMs) on tau peptide.

Post-translational modification (PTM) refers to the covalent and generally enzymatic modification of proteins following protein biosynthesis. Proteins are synthesized by ribosomes translating mRNA into polypeptide chains, which may then undergo PTM to form the mature protein product. PTMs are important components in cell signaling, as for example when prohormones are converted to hormones.

Post-translational modifications can occur on the amino acid side chains or at the protein's C- or N-termini (see, e.g., Pratt, Donald Voet et al., (2006). Fundamentals of biochemistry: life at the molecular level (2. ed.), ISBN 978-0-471-21495-3). They can extend the chemical repertoire of the 20 standard amino acids by modifying an existing functional group or introducing a new one such as phosphate. Phosphorylation is a very common mechanism for regulating the activity of enzymes and is the most common post-translational modification (see, e.g., Khoury G A et al., Scientific Reports, 1:90). Many eukaryotic and prokaryotic proteins also have carbohydrate molecules attached to them in a process called glycosylation, which can promote protein folding and improve stability as well as serving regulatory functions. Attachment of lipid molecules, known as lipidation, often targets a protein or part of a protein attached to the cell membrane.

The post-translational modifications (PTMs) identified in the methods described herein can be any type of PTMs. In some embodiments, the PTMs are one or more of phosphorylation, glycosylation, glycation, prolyl-isomerization, cleavage or truncation, nitration, polyamination, ubiquitination, acetylation, methylation, dimethylation, trimethylation or sumoylation. Unless otherwise indicated, all numbering of amino acid residues of tau protein described herein is based on the human 2N4R isoform.

### Identified Cleavage Sites in Tau

It is indicated that the cleavage of Tau by IP promotes fibrillization and seeding. These cleavage sites can be targets for agents such as antibodies to treat the spreading of the disease. The N-Terminal tau that is found in the soluble fractions can also be used to diagnose tau by making antibodies.

AD-specific cleavage sites, specifically the fragments missing from aggregated tau (i.e. the cleaved N and C terminal fragment peptides) could be monitored in biofluids (CSF, serum, plasma) as potential biomarkers. Neoepitope antibodies could be utilized both for therapeutic purposed and antibody-based biomarker assays.

It is identified herein that the S262 phosphorylation plays a role in cleavage. T231 and S235 phosphorylation also play a role in cleavage. Acetylation also plays a role so kinase and acetylase inhibitors are important for the activity of the prions and fragmentation. Removing ubiquitin helps to prevent the spread of prion activity.

Phosphorylation, particularly at Y394, S396, S400, T403, and S404 are located in close proximity to the Y394 (b in manuscript) cleavage site, and have substantial effects on cleavage at this side. Table 2 show examples are known tau cleavage sites.

TABLE 2
Known Tau cleavage sites

| Cleavage | | | | | Current | |
|---|---|---|---|---|---|---|
| site | Enzyme | CTRL | SAD | Reference | study | Aggregation |
| M1-A2 | | V | V | Derisbourg et al | X | |
| A2-E3 | thrombin [137], Calpain-1 | | | 34, 137, 33 | X | |
| | [33] and -2 [34 | | | | | |
| V10-M11 | | V | V | Derisbourg et al | X | |
| M11-E12 | | V | V | Derisbourg et al | V | |
| D13-H14 | Caspase-6 | | | 40 | V | |
| D25-Q26 | Caspase-3 [135] and | | V | 135, 33, 34, 36, 41 | X | |
| | Calpain-1 and 2 | | | | | |
| K44-E45 | Calpain-1 [32] | | | 32 | X | |
| T102-A103 | | V | V | Derisbourg et al | X | |
| T123-Q124 | | V | V | Derisbourg et al | V | |
| Q124-A125 | thrombin [137], Calpain-1 | V | V | | X | |
| | [33] and -2 [34 | | | | | |
| R126-M127 | | V | V | Derisbourg et al | X | |
| K150-I151 | | | | 100 | X | |
| A152-T153 | ADAM10 [78] | | V | 78 | X | |
| R155-G156 | | | | [137] | X | |
| G156-A157 | Thrombin | | | 135 | X | |
| I171-P172 | | V | V | Derisbourg et al | X | |
| A173-K174 | | V | V | Derisbourg et al | X | |
| G196-Y197 | | X | V | Chen et al | X | |
| Y197-S198 | Chymotrypsin [138] | | | | X | |
| R209-S210 | Thrombin | | | [137] | X | |
| P223-K224 | | V | V | Derisbourg et al | X | |
| R230-T231 | Thrombin [137], Calpain-1 | X | V | 137, 33, 34, 139 | X | |
| | [33] and -2 [34] | | | | | |
| S237-S238 | | V | V | Derisbourg et al | X | |
| A239-K240 | | V | V | Derisbourg et al | X | |
| R242-L243 | Calpain-1 [37] | | V | 37 | tryptic | |
| L243-Q244, | | V | V | Chen et al | V | |
| Q244-T245 | | V | V | Chen et al | V | |
| N255-V256 | AEP [80] | V | X | AEP [80]. | X | ↑ |
| S258-K259 | | V | V | Derisbourg et al | X | |
| I260-G261 | | V | V | Derisbourg et al | X | |
| N279-K280 | | V | V | Derisbourg et al | V | |
| G304-S305 | | X | V | Chen et al | V | |
| S305-V306 | | V | V | Derisbourg et al | V | |
| Q307-I308 | | V | V | Derisbourg et al | V | |
| I308-V309 | | V | V | Derisbourg et al | V | |
| Y310-K311 | | V | V | Derisbourg et al | X | |
| G323-S324 | | V | V | Chen et al | V | |
| G326-S326 | | V | V | Chen et al | V | |
| D314-L315, | Caspase-2 [42] | | | 42 | V | |
| H330-K331 | | V | V | Derisbourg et al | X | |
| N368-K369 | AEP [80] | X | V | AEP [80]. | V | ↑ |
| E391-I392 | | X | V | Derisbourg et al, [symbol] | X | |
| Y394-K395 | | | | Derisbourg | V | |
| D402-T403 | Caspase-6 [43] | | V | 55, 57 | V | ↑ |
| D421-S422 | Caspase-1, -3, -6, -7 | X | V | 44 | V | |
| | and -8 [44] | | | | | |

[symbol]

indicates data missing or illegible when filed

In some embodiments, the agent targets one or more cleavage sites of Tau, thereby inhibiting the cleavage of Tau by IP and the fibrillization and seeding of tau peptides.

### Inhibition of Cytokines or Cytokine Receptors

Inhibition of pathways such as cell stress signaling pathway, or inflammatory pathways using inhibitors such as STAT inhibitors or antibody treatments to the cytokines discovered is key to preventing the spread and progression of tauopathies. For example, inhibiting cytokines using antibodies and inhibitors to all the cytokines such as IL6, OSM (IL6 family), IL1B, II15, IFN-γ, and TNF-α and as well as inhibitors to transcription factors STAT1/3, SMARCA4, and IRF2. In some embodiments, cytokine receptors are blocked with antibodies or with small molecules.

In some embodiments, the agent decreases the expression and/or activity of a cytokine or a cytokine receptor. In some embodiments, the cytokine is selected from the group consisting of IL-6, OSM (IL6 family), IL-1B, IL-15, IFN-γ, and TNF-α.

In some embodiments, the agent decreases the expression and/or activity of a transcription factor. In some embodiments, the transcription factor is selected from the group consisting of STAT1/3, SMARCA4, and IRF2.

### Examples

The invention is further described in the following examples, which do not limit the scope of the invention described in the claims.

### Methods

The following materials and methods were used in the following examples.

### Sarkosyl Fractionation of Post-Mortem Tissue and Preparation for LC/MS

Data for semitryptic peptide analyses were generated from a recently published set of MS data (Wesseling et al, 2020). For this data set, human post-mortem parietal cortex (Brodmann area, BA39—angular gyrus) specimens from AD patients and non-demented age-matched controls were obtained from 5 different brain banks: 1) the Neurodegenerative Disease Brain Bank (NDBB), Memory and Aging Center, University of California, San Francisco (UCSF), CA; 2) the University of Maryland Brain & Tissue Bank at the University of Maryland School of Medicine, Baltimore, MD; 3) the Harvard Brain Tissue Resource Center, McLean Hospital, Harvard Medical School, Belmont, MA; 4) the University of Miami (UM) Brain Endowment Bank, Miller School of Medicine, Miami, MD; 5) the Human Brain and Spinal Fluid Resource Center (HBSFRC), VA West Los Angeles Healthcare Center, Los Angeles, CA. Tissue from brain banks 2) to 5) were acquired through the NIH NeuroBioBank (U.S. Department of Health and Human Services, National Institutes of Health). Pathological and clinical information, if available, were de-identified. To isolate SI Tau, 0.25-0.35 g sections of cortical brain specimens were homogenized in 5 volumes lysis buffer (25 mM Tris-HCl buffer, pH 7.4, containing 150 mM NaCl, 10 mM ethylene diamine tetraacetic acid (EDTA), 10 mM EGTA, 1 mM DTT, 10 mM nicotinamide, 2 μM trichostatin A, phosphatase inhibitor cocktail (Sigma), and protease inhibitor cocktail (Roche)), using a Precellys® bead beater and clarified by centrifugation at 11,000×g for 30 min at 4° C. Lysates were extracted with 1% sarkosyl for 60 min at 4° C. and ultra-centrifuged at 100,000×g for 2 h at 4° C. The soluble supernatant was removed, and the sarkosyl insoluble fraction was solubilized in 1% SDS. Sarkosyl soluble (SS) and insoluble Tau fractions were diluted with 8 M urea and processed separately using filter-aided sample preparation (FASP) (see, e.g., Wisniewski, J. R., Zougman, A., Nagaraj, N. & Mann, M. Universal sample preparation method for proteome analysis. Nat Methods 6, 359-362, doi: 10.1038/nmeth. 1322 (2009)). Protein mixtures were digested with 12.5 ng/μl trypsin (sequencing grade modified trypsin, Promega, Madison, WI) overnight at 37° C. Acidified peptides were desalted using C18 extraction plates (Waters). Vacuum-dried peptides were reconstituted in sample buffer (5% formic acid, 5% acetonitrile (ACN)) containing indexed retention time (iRT) peptides (Biognosys).

### Preparation of LMW/HMW Tau for LC/MS

Mass spectrometry analyses on High Molecular Weight (HMW) and Low Molecular Weight (LMW) was performed as published previously (ref). HMW was prepared as previously described (see, e.g., Takeda, S. et al. Neuronal uptake and propagation of a rare phosphorylated high-molecular-weight tau derived from Alzheimer's disease brain. Nat Commun 6, 8490, doi: 10.1038/ncomms9490 (2015)). Briefly, 300-500 mg frozen brain tissue from the frontal cortex of four patients with AD and four NDC subjects were homogenized in 5× volumes of cold PBS (+protease inhibitor), which was centrifuged at 10,000×g for 10 min at 4° C. The supernatants (10,000 g extract) were fractionated by molecular weight using SEC using an AKTA purifier 10 (GE Healthcare). Fractions were collected every 1 min (0.5 ml/fraction) from 5.5 mL elution volume (Fraction 2) to 16.5 ML (Fraction 20). For MS analysis fraction 3 and 4 were processed for HMW Tau and fraction 14, 16, 18 for LMW Tau. Four volumes (vol/vol) of cold acetone (−20° C.) were added to each fraction to precipitate the protein, followed by vigorous vortex and incubation for 90 min at −20° C. Pelleted protein was collected by centrifugation at 13,000×g for 10 min at 4° C., aspiration of the supernatant, and resuspension of the pellets in 105 μl of PBS. Effective isolation of LMW/HMW Tau species was confirmed by ELISA and western blot (data not shown). Samples were digested with trypsin using the FASP protocol after reduction with TCEP and alkylation using iodoacetamide. Peptide eluates were acidified and desalted using reversed phase C-18 microspin columns (SEMSS18R, Nest Group), vacuum dried and frozen at −20° C. prior to LC-MS/MS analysis.

### Preparation of Soluble MC1-Isolated Tau Filaments for LC/MS

Mass spectrometry analysis on MC1-isolated Tau was performed on previously published data (ref). Briefly, MC1-isolated Tau was obtained from Peter Davis, purified using MC-1 antibody immunoaffinity columns from 3 separate AD patients as previously described¹⁵. Purified MC-1 isolated Tau was digested with trypsin using the FASP method as described above after reduction with DTT and alkylation with 1% acrylamide. Heavy Tau standard peptide was spiked in for FLEXITau experiments. Peptide eluates were acidified and desalted using reversed phase C-18 microspin columns (SEMSS18R, Nest Group), vacuum dried and frozen at −20° C. prior to LC-MS/MS analysis.

### GELFrEE Fractionation

For GELFrEE fractionation, 200 μg of SI Tau was reduced with DTT reducing agent in acetate sample buffer at 50° C. for 10 minutes. The sample was loaded in a channel of a 8% GELFREE cartridge and separated in 16 fractions in a GELFREE 8100 fractionation system over a period of 3 hours, collecting fraction 1-5 running at 50 V, fraction 6-13 at 100 V, and fraction 14 and 15 at 200 V. Aliquots of 2λ10 μL were taken from the 150 μl-200 μL volume of each fraction. An aliquot of 10 μL of each fraction was mixed with sample buffer and ran on a BOLT 4-12% Bis-Tris gel and transferred to a PVDF membrane. The PVDF membrane was stained with LICOR REVERT 700 total protein stain (Licor) and imaged on a Licor Odyssey CLx imaging system. Membranes were then probed with Tau 5 antibody and a donkey anti mouse 800 secondary antibody and visualized on the Licor Odyssey CLx imaging system. The remainder of each fraction was then processed by FASP after reduction with DTT and alkylation with acrylamide using a peptide to trypsin ratio of 1:50. Peptides were dried by vacuum centrifugation, and desalted using Nest C18 columns. Peptides were resuspended in 5% ACN/5% FA MS sample buffer at −20 C until use

### In-Vitro 20S Immunoproteasome Degradation Assays of 0N4R and 2N4R Tau

For the degradation assays, 1.5 μM human recombinant 0N4R Tau (BostonBiochem, Cambridge, MA) and 15 nM human 20S immunoproteasome (Enzo, Farmindal, NY) were incubated for 60 minutes at 37° C. in activation buffer. Analog, 2.0 μM human recombinant 2N4R Tau (BostonBiochem, Cambridge, MA) and 20 nM human 20S immunoproteasome were incubated for 45 minutes at 37° C. in activation buffer. The degradation reactions were halted by denaturation (70° C. for 10 minutes) and Tau fragments as well as immunoproteasome subunits were resolved on a BOLT 4-12% Bis-Tris gel. The illustration of the bands was achieved by silver staining following the vendor's protocol (Pierce™ Silver Stain for Mass Spectrometry, Thermo Fisher Scientific), the lanes were cut into 8 individual fractions and further processed for mass spectrometry.

### In-Gel Digest of In-Vitro 20S Immunoproteasome Degradation Assays

The gel fractions were cut into smaller pieces, distained according to vendor's protocol (Pierce™ Silver Stain for Mass Spectrometry, Thermo Fisher Scientific) and dehydrated by the addition of acetonitrile (˜3 times the voluminal of gel pieces) and incubation of 10 minutes at room temperature. During this procedure, the gel pieces shrunk and turned to an opaque-white color. Afterwards, the acetonitrile was removed, and gel pieces were air-dried (˜5 minutes) before proceeding to the reduction-alkylation reaction of cysteine residues. For the reduction, gel pieces were covered with a solution of freshly prepared 40 mM β-mercaptoethanol/50 mM ammonium bicarbonate and agitated for 45 minutes at 55° C. After that, the reducing solution was replaced with the same amount of 55 mM acrylamide in 50 mM ammonium bicarbonate and incubated for 30 minutes at room temperature. To terminate the alkylation step, the acrylamide solution was removed, the gel pieces were washed twice under agitation for 10 minutes in 50 mM ammonium bicarbonate followed by dehydration in acetonitrile upon turning opaque-white in color (˜5 minutes). Air-dried gel pieces were covered with 50 mM ammonium bicarbonate containing 12.5 ng/μl trypsin (Promega, Madison, WI) and allowed to swell for 60 minutes on ice. After rehydration, excess trypsin solution was removed and gel pieces were covered with 50 mM ammonium bicarbonate to ensure their immersion throughout digestion for 16 hours at 37° C. The supernatant was transferred to a new tube, the gel pieces were washed twice for 10 minutes with 300 μl of 2:1 (v/v) 50 mM ammonium bicarbonate/acetonitrile each and combined with the other supernatant. Acetonitrile was removed under reduced pressure, samples were concentrated to ˜150 μl (Vacufuge plus, Eppendorf), acidified with formic acid (0.5%), desalted on NEST MicroSpin C18 spin columns (The Nest Group), concentrated to dryness (Vacufuge plus, Eppendorf) and resuspended in 5% formic acid/5% acetonitrile loading buffer for LC-MS/MS.

Cell lysates preparation, SDS-PAGE and Immunoblotting HEK293T cells were harvested in cold 0.01M PBS and lysed in cold lysis buffer ((50 mM TrisHCl, 150 mM NaCl, 1 Mm EDTA, 1% NP40, pH 6.8) supplemented with protease and phosphatase inhibitor. After incubation on ice for 30 mins, cell lysates were clarified by centrifugation at 12,000 rpm for 15 mins. Clarified cell lysates were denatured and were run on a BOLT 4-12% Bis-Tris gels in duplicate. One gel was stained with Simply Blue Safestain and one gel was transferred to a PVDF membrane. The PVDF membrane was stained with LICOR REVERT 700 total protein stain (Licor) and imaged on a Licor Odyssey CLx imaging system. Membranes were then probed with GFP Antibody (B-2) Alexa Fluor® 680 (Santa Cruz Biotechnology), and subsequently with 4R Tau antibody (Millipore Sigma) followed by IRDye®: 800CW Goat anti-Rabbit antibody (Licor). Membranes were subsequently scanned on the Odyssey CLx imaging system. For immunoproteasome knockdown experiments, proteins were instead transferred to nitrocellulose membranes (Invitrogen, Carlsbad, California) and membranes were blocked with 5% non-fat-milk in 0.01M PBST (0.1% Tween20) at RT for 1 hr. Membranes were then incubated overnight at 4° C. with primary antibodies diluted in 0.01M PBST as follows: rabbit anti-PSMB 10 (1:2000, GenTex), Rabbit anti-PSMB 9 (1:1000, GenTex), Rabbit anti-PSMB 8 (1:1000, Abcam), Rabbit-anti GAPDH (1:5000, Cell Signalling). Following several washes with PBST, membranes were incubated with goat anti-rabbit (1:5,000, Calbiochem) at RT for 1 hr. Following several washes with PBST, Chemiluminescent substrate (Amersham ECL Plus, GE Healthcare Life Sciences) was added, and images were captured real-time at 10-20 sec intervals using a FujiFilm LAS-3000 with CCD camera (GE Healthcare Life Sciences). Gel fragments were then cut out based on Tau immunoreactivity and processed for LC/MS analysis. Gel fragments were reduced with 20 mM DTT, alkylated with 1% acrylamide, and digested with 100 μL 100 mM ABC containing 100 μL of 12.5 ng/μl trypsin overnight. Peptides were collected by washing with 100 mM ABC, 100 μL NaCl, and dehydrated in 100% ACN. Peptides were dried by vacuum centrifugation and desalted using Nest C18 columns. Peptides were resuspended in 5% ACN/5% FA MS sample buffer and frozen at −20 C until use.

### LC-MS/MS Measurements

Sarkosyl-insoluble samples of 46 AD patients and 44 matched control individuals were analyzed using a analyzed using a Q Exactive HF and QE mass spectrometer (Thermo Fisher Scientific, Bremen) coupled to a micro-autosampler AS2 and a nanoflow HPLC pump (Eksigent, Dublin, CA). Peptides were separated using an in-house packed C18 analytical column (Magic C18 particles, 3 cles, 3 art Michrom Bioresource) or a PicoChip column (150 μm×10 cm Acquity BEH C18 1.7 μm 130 Å, New Objective, Woburn, MA) column over a linear 120 min gradient starting from 95% buffer A (0.1% (v/v) formic acid in HPLC-H2O) and 5% buffer B (0.2% (v/v) formic acid in acetonitrile) to 35% buffer B. For LMW/HMW Tau, randomized samples (SEC fractions 2,3,4 and 14,16,18 for 4 AD and 4 control human brain samples) were analyzed in duplicates using a Q Exactive™ mass spectrometer (Thermo) coupled to a micro-autosampler AS2 and a nanoflow HPLC pump (Eksigent). For data-dependent acquisition experiments LC-MS/MS analysis, peptides were loaded on a capflow PicoChip column (150 μm×10 cm Acquity BEH C18 1.7 μm 130 A, New Objective, Woburn, MA) with 2 μl/min solvent A. The proteolytic peptides were eluted from the column using a 60 min gradient starting at 2% solvent B (0.1% FA) in solvent A, which was increased to 35% at a flowrate of 1 μl/min. The PicoChip containing an emitter for nanospray ionization, which was kept at 50° C. and mounted directly at the inlet to the HF mass spectrometer. The mass spectrometer was operated in positive DDA top 20 mode with the following MS1 scan settings: mass-to charge (m/z) range 300-1650, resolution 60,000@ m/z 400, AGC target 3e6, max IT 20 ms. MS2 scan settings: resolution 30000 @ m/z 400, AGC target 1e5, max IT 25 ms, isolation window m/z 1.4, NCE 27, charge state exclusion unassigned, 1, >8, peptide match preferred, exclude isotopes on, and dynamic exclusion of 20s. The MC1-immunopurified PHF Tau samples were run using the same parameters as the LMW/HMW Tau on the Q Exactive MS.

Analysis of In-Vitro 20S Immunoproteasome Degradation samples was performed as follows. Samples in LC-MS/MS sample loading buffer were transferred to autosampler vials and analyzed by nLC-MS/MS on nanoElute system (Bruker, Germany) coupled to a timsTOF Pro mass spectrometer (Bruker, Germany) equipped with a CaptiveSpray source. Peptides were then separated by reverse phase liquid chromatography on commercially-packed IonOpticks column (Aurora Series with CaptiveSpray Insert, 1.6 μm C18, 250 mm×75 μm) at a flow rate of 400 nl/min over a 40-minute gradient (Mobile Phase A: 98% H2O, 2% ACN, 0.1% FA, Mobile Phase B=100% ACN, 0.1% FA: 2-37% Mobile Phase B) and a column temperature set to 50° C.

For each run, the column was first equilibrated with 4 column volumes of 100% mobile phase A and then sample was loaded; both steps were performed at 900 bar. After the gradient was completed, the concentration of mobile phase B was ramped up to 80% over the course of 10 minutes and then kept constant at 80% for 10 minutes.

For the MS analysis of the peptides, the timsTOF Pro was operated in PASEF mode and the TIMS enabled using Bruker's default method “DDA PASEF-low_sample_amount_1.9 sec_cycletime”. Briefly, the parameters were as follows: mass range 100-1700 m/z and mobility range 1/K₀ 0.6-1.6 V·s/cm² with a corresponding ramp time of 166 ms and lock duty cycle to 100%. The source was put to CaptiveSpray with the capillary voltage set to 1500 V, the dry gas to 3.0 l/min and the dry temperature to 180° C. The parameters for PASEF were set to 10 MS/MS scans with a total cycle time of 1.88 s, charge range 0-5, active exclusion for 0.4 minutes (precursors were reconsidered within that time frame if the current intensity was at least 4-fold of the previous one), target intensity of 20,000 and intensity threshold of 1,000. The collision energy was adjusted to the mobility of the ions ranging from 20 eV for 1/K₀ 0.6 V·s/cm² up to 59 eV for 1/K₀ 1.6 V·s/cm². The conversion into MGF file format was facilitated with Bruker Compass DataAnalysis Version 5.2.

### Identification of Semitryptic Peptides and Analyses of Differential Expression

Raw data derived from the Q Exactive/Q Exactive HF or the timsTOF Pro were analyzed with MaxQuant Version 1.6.6.077 or 1.6.10.43, respectively, using an in-house database for SS and SI datasets, and the Uniprot database including isoforms for MC1-isolated Tau, LMW/HMW Tau, and the in-vitro Tau degradation assays. For the FL Tau biosensor experiments, the Uniprot database including isoforms was modified by adding entries for the fusion protein construct. For differential expression analysis, databases with reviewed entries only and no isoforms were used, while for semitryptic database searches databases with reviewed entries and isoform entries were used instead. For data acquired on the Orbitrap systems mass tolerances for the first search were set to 20 ppm and for the second search to 4.5 ppm, while for the TIMS-DDA first and main search peptide tolerance were decreased to 30 ppm: other “Type”-specific settings were kept at default. The digestion mode was set to specific trypsin (one tryptic site) with up to two missed cleavages for the differential expression analysis, whereas for the semitryptic searches digestion mode was switched to semispecific (one tryptic site). Propionamide in cysteine (+71.0371137878 Da) was set as a fixed modification, whereas oxidation of methionine (+15.9949146221 Da) and acetylation of the N-terminus (+42.0105646863 Da) were variable modifications. For all other search parameters, the default settings were used. Identified peptides with a prior amino acid that was not K or R were designated as N-terminal semitryptic peptides, and peptides not ending in K or R were designated as C-terminal semitryptic peptides. Correct identification was manually validated, and only samples where peptides were identified by MS/MS were used in population frequency counts and the Tau degradation assay. All peptides found by MaxQuant were exported as .txt files, normalized and filtered in excel. Subsequent data illustration was performed in R Studio (v3.6.3) utilizing pheatmap package (version 1.0.12).

### FLEXITau Measurements

LC-SRM measurements of Tau L/H peptide ratios were performed as described previously (REF). The FLEXITau SRM assay quantified an in house list of validated transitions for unmodified Tau peptides that are highly reproducible with low CV. Peptide mixtures were analyzed on a triple quadrupole mass spectrometer (5500 QTRAP, Sciex) using a micro-autosampler AS3 and a nanoflow UPLC pump (both Eksigent/Sciex), using the trap-elute chip system (cHiPLC nanoflex, Eksigent). Briefly, peptides were first loaded onto the trap-chip (200 μm×75 μm, ChromXP C18-CL 3 μm 120 A, Nano cHiPLC Eksigent) and then separated using a 120 min gradient from 95% buffer A (0.1% (v/v) formic acid in HPLC-H2O) and 5% buffer B (0.2% (v/v) formic acid in ACN) to 35% buffer B on the analytical column-chip (75 μm×15 cm, ChromXP C18-CL 3 μm 120 A, Nano cHiPLC Eksigent). The retention time window was set to 5 min and total scan time to 1.2 s, which ensured a dwell time over 20 ms per transition. To avoid sample carry over, blanks were analyzed between every SRM run. Samples were run in randomized order, with 3 technical replicates per sample. SRM data were analyzed and validated in Skyline (version 2.6, MacCoss Lab Software, University of Washington, Seattle, WA) (see, e.g., MacLean, B. et al. Skyline: an open source document editor for creating and analyzing targeted proteomics experiments. Bioinformatics 26, 966-968, doi: 10.1093/bioinformatics/btq054 (2010)). Only peptides quantified in every sample were included in downstream analysis yielding a final peptide list consisted of 17 Tau peptides. To calculate relative abundance of peptides, the L/H ratio of peak areas for each peptide was divided by the average of the three Tau peptides with the highest ratio, representing the unmodified peptides with the highest relative abundance. FLEXITau is an MS-based strategy that is based on the addition of a heavy full-length 2N4R Tau protein to each sample prior to digestion. Because all heavy Tau peptides are present at equimolar amounts, the ratio of light to heavy peptide can be used to infer the extent of modification/enrichment for each peptide. In brief heavy Tau standard was expressed in a cell-free wheat germ expression system containing a full complement of amino acids, with heavy isotope (i.e. 13C and 15N) labeled lysine, arginine and aspartate. The standard was purified using Ni-Sepharose beads (Ni-Sepharose High Performance resin, GE Healthcare). Heavy Tau standard was digested along with samples and spiked in to reach a L/H ratio of approximately 1:1.

### Constructs and Lentivirus Production

To generate recombinant Tau fragments, cDNA for each Tau fragment was inserted into addgene plasmid #29663 with HiFi assembly. The resulting vector encodes a fusion protein consisting of a hexahistidine tag followed eGFP followed by a tobacco etch virus (TEV) enzymatic cleavage site attached to the desired fragment of Tau. The plasmid was transformed into DH5a, and BL21DE3 cells.

The lentiviral plasmid constructs were made by Genecopoeia, Inc. In brief, open-reading frame of gen coding for human Tau isoform 0N4R (NCBI accession No. 016834.4) with P301S mutation was subcloned into pReciever-Lv vector with CMV promotor and c-terminus fused to EYFP. The lentiviruses were made by viral core at Boston Children's Hospital. In brief, transfer plasmid was co-transfected with lentiviral packaging plasmids into HEK293T cells using PEI max. 3 days after transfection, 72 ml virus containing culture medium was cleared by centrifugation at 1500 rpm for 5 min and then passed through a 0.45 μm filter. Concentration of lentivirus using ultracentrifugation was performed with a XE90 Beckman Coulter centrifuge using a SW32Ti rotor. Centrifugation was performed for 2 h at 18000 rpm. Supernatant was completely removed, and virus pellets were resuspended gently in 150 μl DPBS with 0.001% F68. 10 μl aliquots were made and stored at −80° C. till use. Protein expression

The 8 fragments defined by combinations of the N-terminal and C-terminal cleavage sites were produced by recombinant expression in E. coli. Each fragment was named with a number followed by a letter, with increasing number (1-4) corresponding to a shorter N-terminus, and a/b corresponding to the N368-K369 and Y394-K395 cleavages, respectively.

E. coli BL21 (DE3) cells were transfected with constructed DNA variants and stored as frozen glycerol stock at −80° C. Cells from glycerol stock were grown in 10 mL luria broth (LB, Sigma Aldrich, L3022) overnight and then used to inoculate 1 L of fresh LB. Growth of cells were performed at 37° C., 200 rpm with addition of 10 μg/mL kanamycin (Fisher Scientific, BP906) until optical density at 2=600 nm reached 0.6-0.8. Expression was induced by incubation with 1 mM isopropyl-β-D-thiogalactoside (Fisher Bioreagents, BP175510) for 3 hr. Cells were harvested with centrifugation at 5000 g for 30 min. Harvested cells were resuspended in lysis buffer (Tris-HCl, pH=7.4, 100 mM NaCl, 0.5 mM DTT, 0.1 mM EDTA) added with 1 Pierce protease inhibitor tablet (Thermo Scientific, A32965), 1 mM PMSF, 2 mg/mL lysozyme, 20 μg/mL DNase (Sigma, DN25) and 10 mM MgCl₂ (10 mM), and incubated on ice for 30 min. Samples were incubated at 30° C. for 20 minutes, then centrifuged at 10,000 rpm for 10 min to remove cell debris. 1 mM PMSF was added again and the resulting supernatant was incubated for at least 4 h with Ni-NTA resins pre-equilibrated in buffer A (20 mM sodium phosphate, pH=7.0, 500 mM NaCl, 10 mM imidazole, 100 μM EDTA).

The resin was loaded to a column and washed with 20 mL of buffer A, 25 mL buffer B (20 mM sodium phosphate, pH=7.0, 1 M NaCl, 20 mM imidazole, 0.5 mM DTT, 100 UM EDTA). Purified protein was eluted with 15 mL of buffer C (20 mM sodium phosphate, pH=7.0, 0.5 mM DTT, 100 mM NaCl, 300 mM imidazole), or until the resin no longer contained traces of GFP visible. The protein was concentrated to a volume of 2.5 mL and was buffer exchanged into TEV buffer (50 mM Tris buffer, pH7, 100 mM NaCl, 50 mM CaCl₂)) to remove the imidazole, and prepare the protein for TEV cleavage. 35 μL of TEV (˜1 mg/mL stock) prepared in house was added to the solution and incubated at 4° C. overnight with gentle rotation.

To separate each fragment from GFP and TEV a cation exchange (GE CMFF sepharose column) separation followed by size exclusion chromatography using a Biorad superdex70 column. Cation exchange was conducted at pH 8 with a gradient of 0-1 M NaCl elution profile. All peaks showing a UV-Vis signal were collected, and were concentrated to 250 μL. The SEC column was preequilibrated with 2 column volumes of working buffer, and the protein sample was loaded onto the sample loop. 0.5 mL fractions were collected and run on an SDS page gel stained with coomasie blue to determine the fractions containing Tau. All fractions containing Tau were pooled and concentrated to approximately 200 μM as determined by the UV-Vis absorption at 274 nm.

### Recombinant Tau Fibrillization

Fibrils of recombinant Tau fragments used in Tht and TEM assays were generated with 50 μM total protein concentration. 12.5 μM Heparin (Galen laboratory supplies, HEP001) was used for a 4:1 molar ratio of Tau; heparin. Samples were incubated at 37° C. in 384-well Corning plates. Fibrils used in HEK293T seeding assays were formed at 120 μM protein concentration, with 30 μM Heparin.

### In Vitro Seeding and Thioflavin T Experiments

Seeds were generated following the procedure described above at 50 μM protein concentration. The sample was vigorously pipetted to evenly disperse fibrils, and was added at a 2.5 mol % (1.25 μM protein concentration) to a 50 μM sample of new protein. Aggregation was followed with ThioflavinT assays. Thioflavin T (ThT) assays for Beta-sheet content were conducted with 50 μM protein content, and 20 mM ThT in a 384-well Corning plate. A BioTek synergy2 plate reader was used with temperature control set at 37° C.

### Transmission Electron Microscopy

Transmission Electron Microscopy (TEM) imaging was conducted with a 200 kV FEI Tecnai G2 Sphera Microscope. Samples were blotted onto 200-mesh Formvar copper grids, and were negative stained with uranyl acetate.

### Cell Culture, Lentiviral Transduction, FACS Sorting and Stable Cell Line Generation

For recombinant peptide seeding assays, HEK-293 cells stably expressing mCerulean-K18 (P301L/V337M) or mCerulean-Tau 187 (P301L/V337M) were cultured in DMEM supplemented with 10% FBS, 100 μg/ml penicillin/streptomycin. Cultures were maintained in a humidified atmosphere of 5% CO2 at 37° C.

To generate the full-length Tau biosensor cell lines, HEK293T cells (ATCC) were cultured in DMEM (ThermoFisher Scientific) supplemented with 10% fetal bovine serum and 1% penicillin/streptomycin (ThermoFisher Scientific). The cells were maintained in a humidified incubator at 37° C. supplied with 5% CO2 (v/v) and passaged by 0.25% trypsin/EDTA digestion and dissociation on every other day, and culture mediums were tested for myoblast contamination. To generate a stable cell line, HEK 293T cells were plated on 6-well plate and infected on the next day with lentivirus expressing human Tau-0N4R (P301S) with C-terminus fused EYFP. The medium was changed 1 day after infection and transduced cells were passaged to a 10 cm culture plate. Once YFP signal can be seen in 80˜90% of total cells under fluorescent microscope, the cells were digested and dissociated as single cells by 0.25% trypsin/EDTA, pelleted by centrifugation at 1200 rpm for 3 min, and resuspended in DMEM medium supplemented with 2% fetal bovine serum and 1% penicillin/streptomycin. Then, 1 ml cell suspension containing ˜8×10⁶ cell was subjected to FACS sorting (FACS Aria II flow cytometer, flow cytometry research facility at Boston Children's Hospital), a population of single live cells with YFP signal were collected, and plated on 6-well plate. Two days later, the cells were dissociated as single cells as described above and plated on a 10 cm culture plated at density as extremely low as ˜120 cell in total. After 10 days single cells expanded to a mono-clones, so that multiple clones could be isolated under a microscope and transferred separately to a 96-well plate. Finally, multiple mono-clonal stable cell lines with homogeneously and stable expression of human Tau-0N4R (P301S) EYFP were selected, expanded, and cryopreserved in liquid nitrogen for future study.

### Seeding Assays, Cytokine Treatment and SiRNA Transfection to Cell Culture, Immunofluorescence Cell Staining and Microscopy Imaging

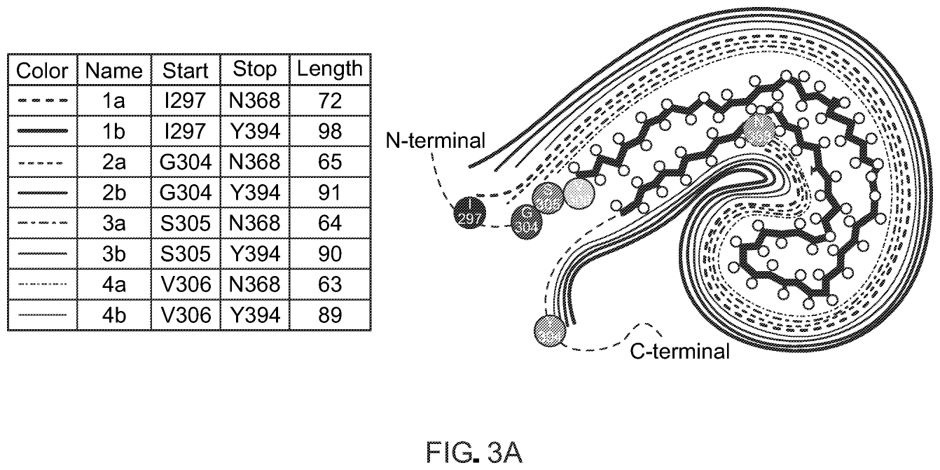

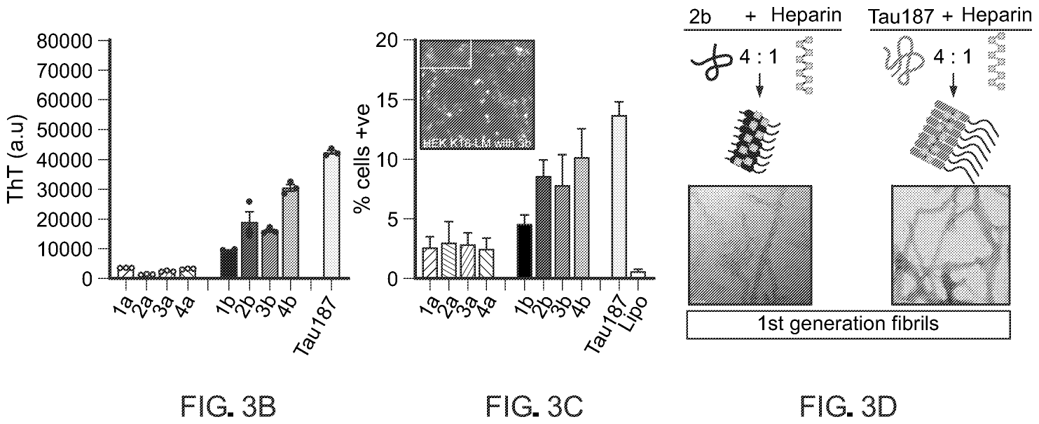

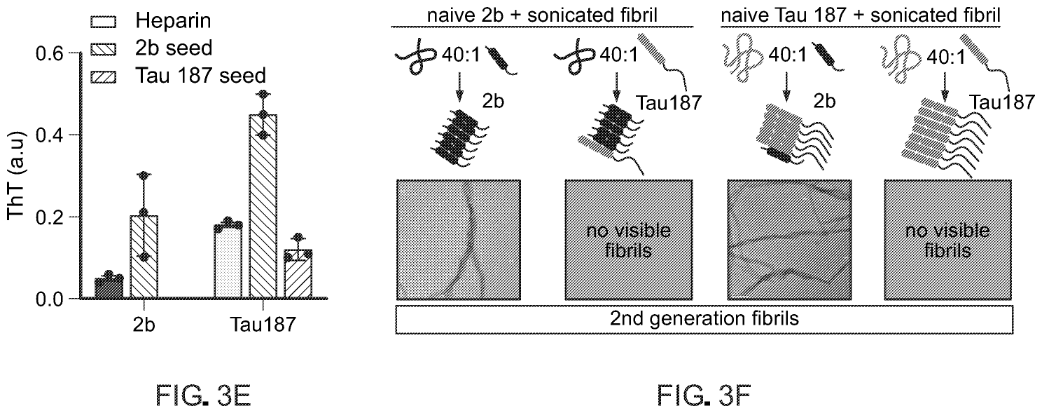

For recombinant peptide seeding experiments, cells were plated in a 96-well plate and the following day Tau species were sonicated 30 s with the micro tip of a Qsonica sonicator and transfected (50 ng/μL final) using Lipofectamine 2000 (Thermo Fisher). For quantification of the assay (FIG. 3C), the number of cells containing one or several puncta were counted and divided by the total number of cells in the well. Each well on a cultured plate provide one point. Quantification results originate from three independent cultures.

The cytokine treatment experiments were performed in mono-clonal cell line stably expressing human Tau-0N4R (P301S) YFP plated in a 96-well plate. Both TNF-α and IFN-γ (Pepro tech) were applied to the culture medium at final concentration of 50˜200 ng/ml for each individual factor, and H2O was used as control to cytokines treatment.

For knock-down experiments, the SiRNA targeting PSMB10 and 9 were ordered from Millipore Sigma. Two different sequences were tested for each target and then one sequence was chosen for final experiments. The MISSION® siRNA Universal Negative Control #1 (SIC001) from Millipore Sigma was used as a negative control to specific targeting SiRNAs. Specific or control SiRNA were transfected by lipofectamine2000 (ThermoFsher Scientific) at final concentration is 200 nM. The cytokines were added to culture medium on the next day after SiRNA transfection.

3˜8 days after treatment, the medium was removed and the cells were fixed by 4% paraformaldehyde in 0.01 M PBS for 15 minutes. Following three washes with 0.01M PBS, cells were permeabilized with 0.15% Triton-X100/0.01 M PBS for 10 minutes, incubated in blocking buffer (8% normal goat serum, 1% BSA, 0.15% Triton-X100 in 0.01 M PBS) for 1 hour at room temperature and followed by incubation with primary antibody, mouse Anti-Tau_R4 (4-repeat isoform RD, clone 1E1/A609) (1:200, EMD Millpore) diluted in blocking buffer. Following 5 washes with 0.01 M PBS, cells were incubated with Alexa Fluor 568 conjugated goat anti-mouse IgG secondary antibodies (1:700, Invitrogen, Carlsbad, California) for 1 hour at room temperature. After 5 washes with 0.01M PBS, cells were counterstained nuclei with DAPI and subjected to imaging. Cell immunostaining in 96-well plate were imaged by a Nikon Ti Eclipse inverted microscope with Coolsnap HQ2 camera (Photometrics, Tucson, Arizona) and Nikon Elements software (Nikon, Melville, New York). Images were acquired as large image stitched with 6×6 fields covering the whole area of a well using a 10× Plan Apo objective to. Data were analyzed using Fuji software (National Institutes of Health) and cropped images were used as representative.

### Statistical Analysis

Data analysis was carried out using a combination of Prism 8, Perseus, Ingenuity Pathway Analysis (IPA), and Cytoscape version 3.7.1 with the ClueGO plugin. For differential expression analysis, protein groups were filtered for contaminants, only identified by site, and reverse hits. Subsequently, the LFQ intensity values were log 2 transformed, and filtered for 70% valid values in at least one treatment group. The remaining 30% of values were imputed from a normal distribution with downshift 1.8 and width 0.3. A t-test with a permutation-based FDR <0.05 was used to determine differentially expressed proteins. Differentially expressed proteins were crossreferenced with the MEROPS peptidase database to identify putative Tau proteases. The whole dataset was also analyzed in IPA, setting the same FDR cut off of 5%, and using the fold change in expression as analysis variable. Putative upstream regulators identified of the type ‘cytokine’ and ‘transcription factor’ were used. For the HEK293 FL Tau biosensor mass spectrometry analysis, proteins that were either detected in 2 out of 3 replicates in one condition were compared by median intensity between groups and proteins changed >5-fold value, or absent in one condition altogether, were included for pathway analysis. Pathway enrichment analysis was performed with Cytoscape with the ClueGo plugin, using the Reactome annotation database. For measurements of individual proteins, median values were used to calculate fold changes.

### Results

Importantly, 1) the identity of the prion-like Tau species, 2) the initiation of the prion-like seed structure 3) and the mechanism by which a prion/seed presumably templates naïve Tau in vitro or in vivo are unknown. Tau is expressed in the brain as 6 different isoforms by alternative splicing and is extensively post-translationally modified, resulting in considerable structural diversity of Tau proteoforms produced from the same gene (see, e.g., Mirbaha, H. et al. Inert and seed-competent tau monomers suggest structural origins of aggregation. Elife 7, doi: 10.7554/eLife.36584 (2018)). Recombinant full-length (FL) Tau does not fibrillize spontaneously in vitro unless a negatively charged co-factor is added. In the absence of an analogous co-factor other molecular-level changes in Tau could result in structural changes that initiate fibril formation observed for AD Tau. While it may be possible that a unmodified full-length Tau protein can simply change its protein fold (see, e.g., Mirbaha, H. et al. Inert and seed-competent tau monomers suggest structural origins of aggregation. Elife 7, doi: 10.7554/eLife.36584 (2018)), overcoming energetic barriers to form Tau seeding positive conformers may require posttranslational modifications including phosphorylation, ubiquitination, and acetylation (see, e.g., Cohen, T. J. et al. The acetylation of tau inhibits its function and promotes pathological tau aggregation. Nat Commun 2, 252, doi: 10.1038/ncomms1255 (2011)) to induce structural changes. Furthermore, a large body of evidence suggests that cleavage of Tau increases aggregation propensity, and different enzymes have been implicated in the pathological cleavage of Tau, including caspases (see, e.g., Rissman, R. A. et al. Caspase-cleavage of tau is an early event in Alzheimer disease tangle pathology. J Clin Invest 114, 121-130, doi: 10.1172/JCI20640 (2004); and Zhao, Y. et al. Appoptosin-Mediated Caspase Cleavage of Tau Contributes to Progressive Supranuclear Palsy Pathogenesis. Neuron 87, 963-975, doi: 10.1016/j.neuron.2015.08.020 (2015)), calpains (see, e.g., Chen, H. H. et al. Calpain-mediated tau fragmentation is altered in Alzheimer's disease progression. Sci Rep 8, 16725, doi: 10.1038/s41598-018-35130-y (2018)), and cathepsins (see, e.g., Kenessey, A., Nacharaju, P., Ko, L. W. & Yen, S. H. Degradation of tau by lysosomal enzyme cathepsin D: implication for Alzheimer neurofibrillary degeneration. J Neurochem 69, 2026-2038, doi: 10.1046/j.1471-4159.1997. 69/052,026.x (1997)). In fact, current cell-based seeding assays mostly rely on the expression of truncated Tau consisting only of the microtubule binding repeat domains (MTBs), a fragment referred to as K18, that additionally includes disease mutations, typically P301L and V337M, precisely because these modifications lower the barrier for their aggregation (see, e.g., Lim, S., Haque, M. M., Kim, D., Kim, D. J. & Kim, Y. K. Cell-based Models To Investigate Tau Aggregation. Comput Struct Biotechnol J 12, 7-13, doi: 10.1016/j.csbj.2014.09.011 (2014)). Moreover, cryo-EM data of Tau fibrils from AD and different Tauopathies show a core domain that contributes to fibril structure, while N and C termini do not.

We show that processing of Tau by immunoproteasomes (IP) can generate aggregation-prone fragments, linking cellular stress and neuroinflammation to initiation of pathology. Using mass spectrometry to study pathological Tau extracted from human AD brain identified AD-specific cleavage sites around the filament core region of Tau and depletion of the N and C-termini. Analysis of changes in abundance of proteins associated with Tau aggregates revealed increases in proteases associated with cell stress and inflammation, including IP subunits that colocalized with pathological Tau in neurons. In vitro, purified IP cleaved recombinant full-length (FL) Tau, and recombinant peptides representing endogenous Tau fragments were able to initiate fibrillization and subsequently seed Tau aggregation in cells. Cytokine-induced IP expression in a FL Tau cell line resulted in Tau cleavage and spontaneous Tau aggregation, which was rescued by knockdown of IP subunits. Together, we propose a model where inflammatory cytokines and cellular stress results in generation of Tau seeds through alterations in Tau processing.

In a previous study, we described changes in isoform composition and posttranslational modifications specific to seed-competent Tau extracted from AD brains (Wesseling et al, 2020). In this study, we perform the first comprehensive analysis of proteolytic cleavage sites for the same cohort, consisting of 49 AD patients and 43 control subjects. We identify consistent AD-specific cleavages and confirm that these cleavages are characteristic of Tau seed competence. We test the in vitro aggregation and seeding competence of representative Tau fragments flanked by high-frequency cleavage sites. Furthermore, we associate the proteolytic cleavage of Tau with stress and inflammation pathways that result in immunoproteosome (IP) expression in neurons in the AD brain. We then validate that the IP links inflammatory signaling to Tau cleavage and spontaneous aggregation in a cell model. The work described here constitutes an important contribution to the molecular characterization of endogenous human Tau fragments in AD, and for the first time provides insight into, the mechanism by which stressors and environmental risk factors produce Tau species capable of initiating aggregation through the activation of inflammatory pathways.

### Proteolytic Cleavage Sites Surround the Aggregation-Prone Core Domain in AD SI Tau

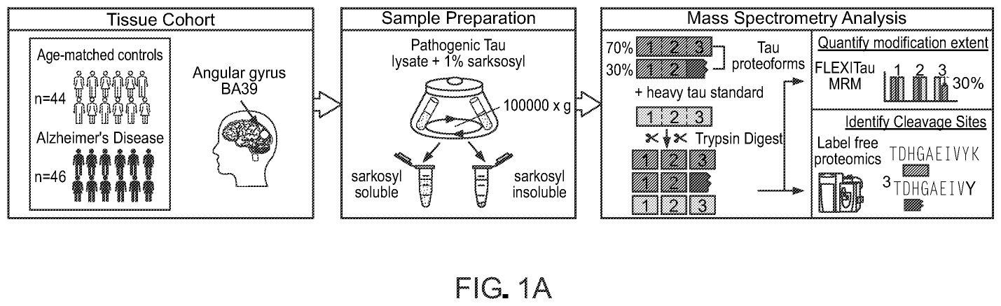

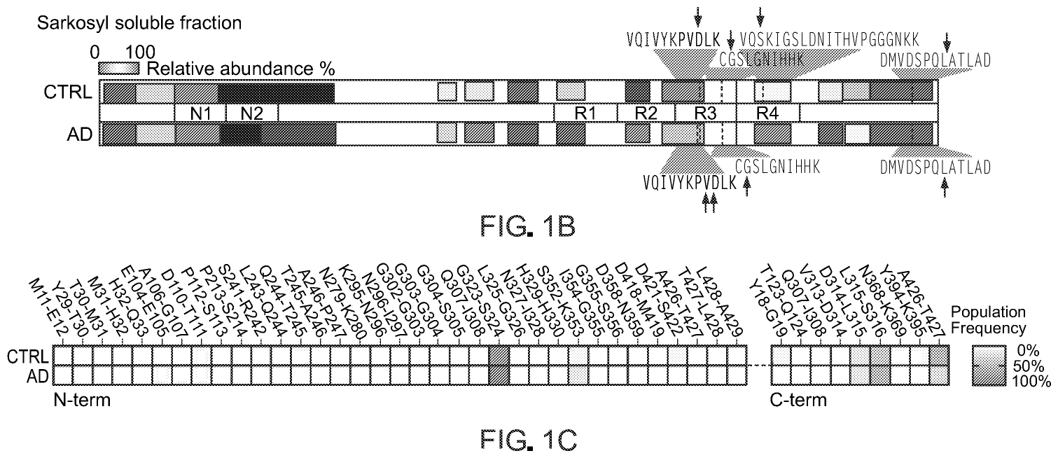

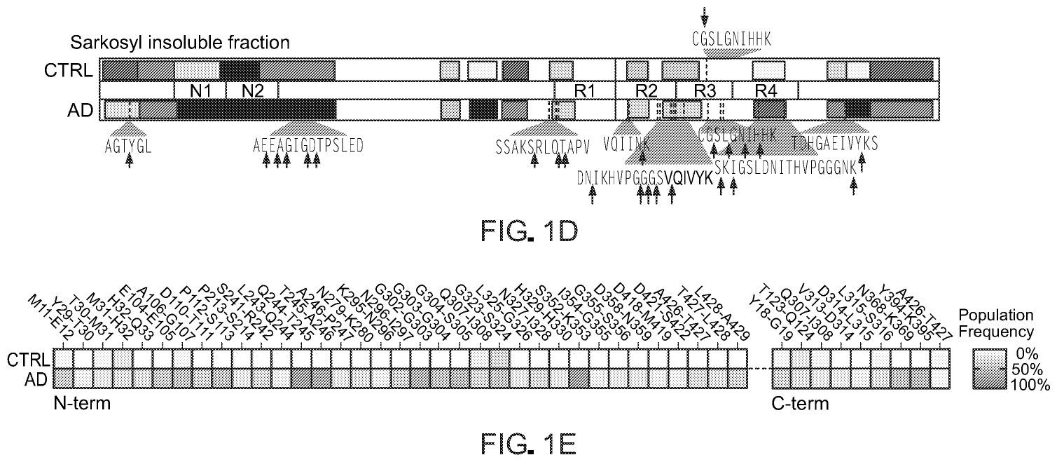

To identify cleavage sites specific for pathological Tau, we analyzed MS data for sarkosyl fractionated tissue lysates extracted from post-mortem brain (BA39) of 47 AD patients and 41 NDCs. The sarkosyl soluble (SS) fraction contains largely monomeric Tau species that are inactive in cell-based Tau seeding assays (non-seeding), while the sarkosyl insoluble (SI) fraction consists of oligomeric and fibrillized tau species (see, e.g., Lasagna-Reeves, C. A. et al. Identification of oligomers at early stages of tau aggregation in Alzheimer's disease. FASEB J 26, 1946-1959, doi: 10.1096/fj. 11-199851 (2012)) that can seed aggregation in cell lines and mouse models (seed-competent) (see, e.g., Narasimhan, S. et al. Pathological Tau Strains from Human Brains Recapitulate the Diversity of Tauopathies in Nontransgenic Mouse Brain. J Neurosci 37, 11406-11423, doi: 10.1523/JNEUROSCI.1230-17.2017 (2017)). We used FLEXITau (Full-Length Expressed Stable Isotope-labeled Tau) (see, e.g., Mair, W. et al. FLEXITau; Quantifying Post-translational Modifications of Tau Protein in Vitro and in Human Disease. Anal Chem 88, 3704-3714, doi: 10.1021/acs.analchem.5b04509 (2016)) targeted MS data to quantify differential peptide abundance across the Tau sequence, and label-free proteomics data to identify specific cleavage sites (FIG. 1A).

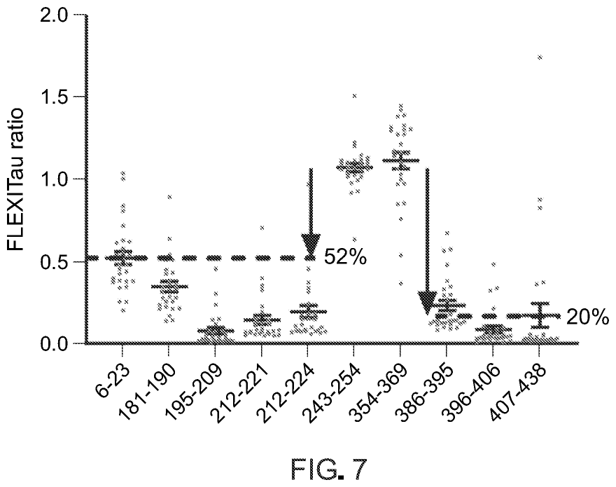

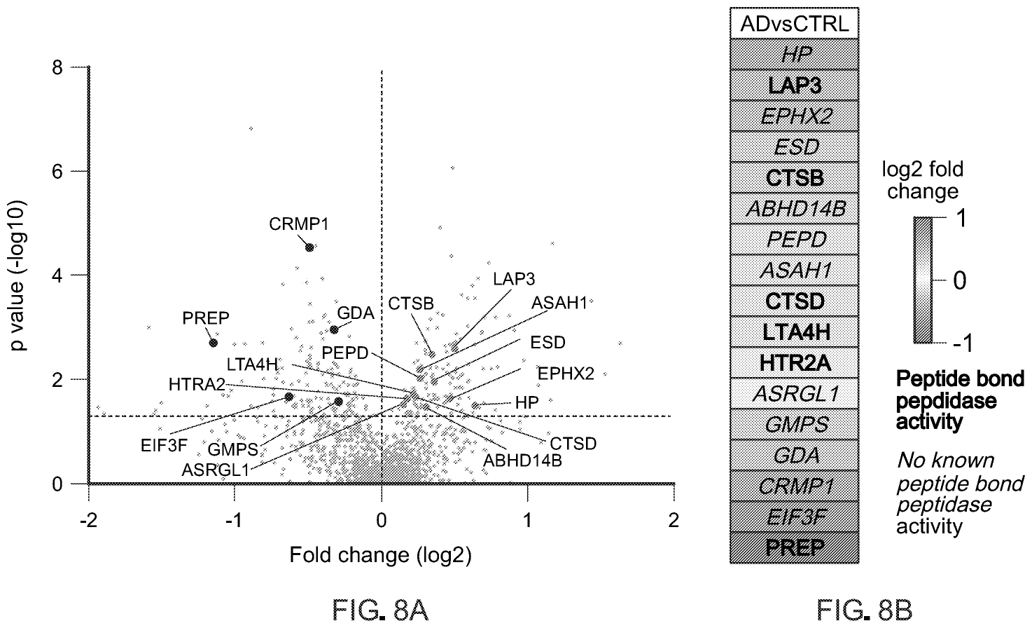

In the SS fraction there were no differences between the AD and NDC groups in the relative unmodified peptide abundances (FIG. 1B). While tau cleavages were detected, the sites and their population frequencies (FIG. 1C) were similar in AD and NDC, confirming that there were no large changes in tau processing in the SS fraction. In the SI fraction (FIG. 1D) on the other hand, there were marked differences between AD and NDC groups. The FLEXITau data revealed depletion of the N and C-terminal peptides by 48% and 80% respectively (FIG. 7) compared to the central region between residue L243 and K369 . . . consistent with cleavages at both the N and C-termini.

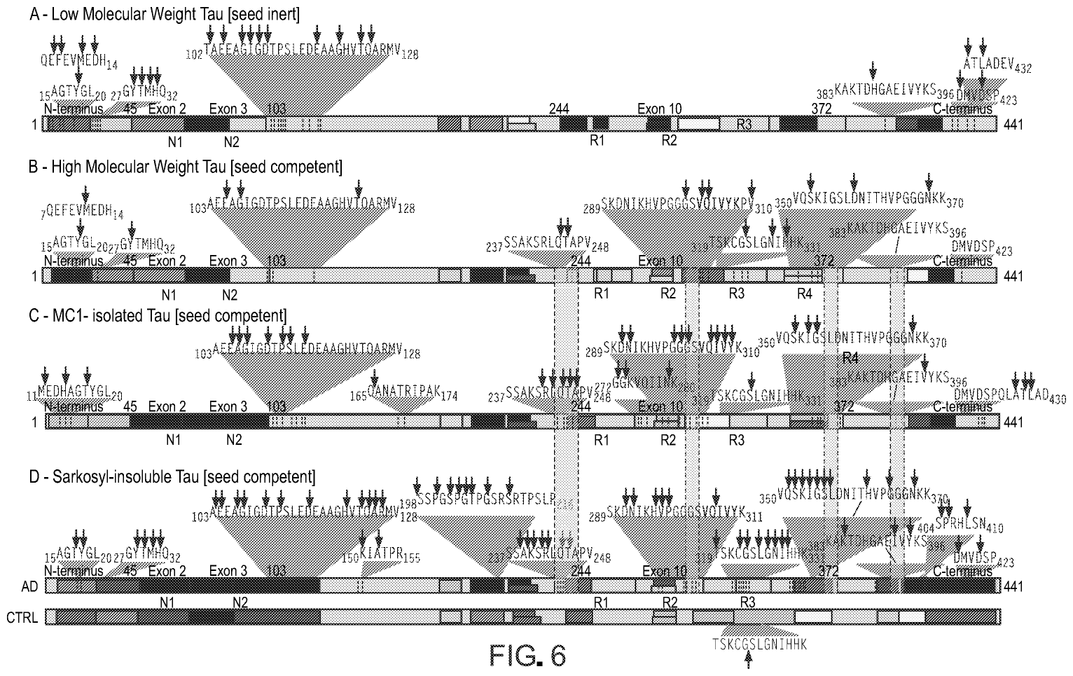

In order to explain this pattern, we searched the label-free data for evidence of proteolytic cleavage. We detected 24 cleavage sites (21N-term vs 3 C-term) with high population frequency (>5 cases) in the AD group whereas only 3 cleavage sites (2 N-term/1 C-term) were found with a frequency >5 cases in the NDC group (FIGS. 1C and 1E). Importantly, high population frequency cleavage ‘hotspots’ identified at N-termini (S241-A246, N296-V306, S352-G355) and C-termini (N368-K369, Y394-K395) of peptides were present in AD but not in NDC SI Tau. To ascertain that the cleavage sites were characteristic of seed-competent tau, we extended our analysis to two other seed competent Tau preparations from AD brain: Tau immunopurified using the MC-1 conformational tau antibody, and high molecular weight (HMW) Tau prepared by size exclusion chromatography (FIG. 6). AD-specific cleavage sites were also observed in both seeding positive preparations, but were not detected in non-seeding low molecular weight AD Tau, showing that specific cleavage site “hot spots” are signatures of seed-competent Tau species

Proteases in the SI Fraction of AD Associated with Inflammatory Signaling Cleave Tau

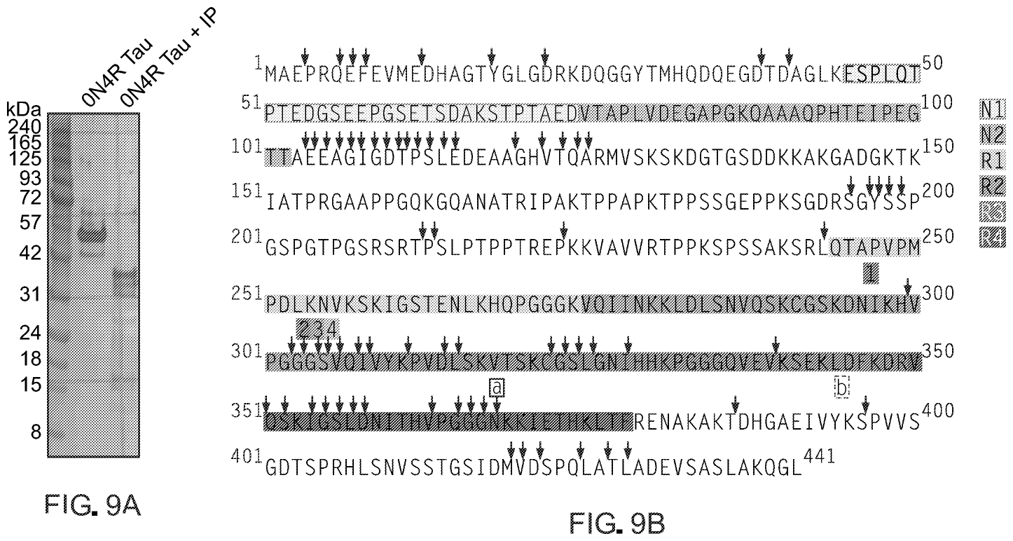

The presence of AD-specific endogenous cleavages dictates that there must be disease-process associated changes in protease expression or activity. To identify such changes, we analyzed the differential protein abundance in the SI fraction. We focused on the SI fraction because it is enriched in proteins associated with Tau aggregates, and therefore represents proteins from neurons with Tau pathology. Next we cross-referenced the up/down-regulated proteins with the MEROPS peptidase database (see, e.g., Rawlings, N. D. et al. The MEROPS database of proteolytic enzymes, their substrates and inhibitors in 2017 and a comparison with peptidases in the PANTHER database. Nucleic Acids Res 46, D624-D632, doi: 10.1093/nar/gkx1134 (2018)) to identify differentially abundant proteins with annotated protease activity (FIG. 3B). There were 6 increased proteins and 5 decreased proteins with annotated protease activity (FIG. 3B), including three subunits of the immunoproteasome, PSMB8 and 9 PSMB10. Out of the decreased proteins in the SI, 3 were deubiquitinases, consistent with the known dysregulation of the ubiquitin-proteasome system in AD (see, e.g., Sulistio, Y. A. & Heese, K. The Ubiquitin-Proteasome System and Molecular Chaperone Deregulation in Alzheimer's Disease. Mol Neurobiol 53, 905-931, doi: 10.1007/s12035-014-9063-4 (2016)). The levels of the upregulated proteases were examined for correlation with the intensity of cleavage-specific peptides in each sample to determine if any cleavage sites could be associated with protease abundance (FIG. 9). PSMB9 levels correlated with cleavages G303-G304 and G304-S305 N-terminal to the MTBR, whereas PSMB8 correlated with cleavage at Y394-K395 on the C-terminal side. The other proteases were not significantly correlated with any sites of interest.

To understand which pathways could result in increased abundance of specific proteases in the insoluble fraction, pathway enrichment analysis was performed on the upregulated proteins. The top enriched Reactome pathways included immune system and cellular response to stress pathways, (FIG. 3C). The PSMB 8 and 9 subunits are part of the immune system and cellular response to stress gene sets, suggesting that changes in these pathways may drive the increase in IP abundance.

Considering the clear enrichment of inflammatory pathways, we evaluated if there was evidence for cytokine signatures associated with the protease abundance changes. The inflammatory cytokines IL6, OSM (IL6 family), IL1B, 1115, IFNG, and TNF-α as well as transcription factors STAT1/3, SMARCA4, and IRF2 (FIG. 3D) were increased, suggesting the protease changes may be explained by a local inflammatory signaling environment. The consistent fold change in the IP subunits and its well-established role in proteolysis under inflammatory or stress conditions (see, e.g., Seifert, U. et al. Immunoproteasomes Preserve Protein Homeostasis upon Interferon-Induced Oxidative Stress. Cell 142, 613-624, doi: 10.1016/j.cell.2010.07.036 (2010)) focused our attention on the hypothesis that increased IP activity contributes to Tau cleavage in AD.

The IP is not expressed in appreciable amounts in neurons under normal conditions, and we therefore examined IP subunit expression in in postmortem AD tissue. Previous studies have described glial expression of the IP (see, e.g., Mishto, M. et al. Immunoproteasome and LMP2 polymorphism in aged and Alzheimer's disease brains. Neurobiol Aging 27, 54-66, doi: 10.1016/j.neurobiolaging.2004.12.004 (2006); and Orre, M. et al. Reactive glia show increased immunoproteasome activity in Alzheimer's disease. Brain 136, 1415-1431, doi: 10.1093/brain/awt083 (2013)), however our studies suggest a neuronal IP expression given that the AD SI proteins originate from neurons with Tau pathology. We performed multiplexed immunostaining on frontal cortex tissue sections to visualize the neurons (NeuN), along with PSMB8/9/10 and two pTau epitopes (AT180-FIG. 3E). No pTau or neuronal IP expression was observed in CTRL brain tissue sections. Strikingly, in AD brain tissue sections PSMB8 and 9 immunoreactivity colocalized with pTau inclusions in neurons, both in neurofibrillary tangles and neuropil threads. While PSMB10 expression was also evident in neurons, it did not appear to colocalize with phospho-Tau. These data clearly show that PSMB8/9 immunoreactivity colocalizes with Tau pathology in neurons, supportive of our proposed mechanism of IP cleavage of Tau

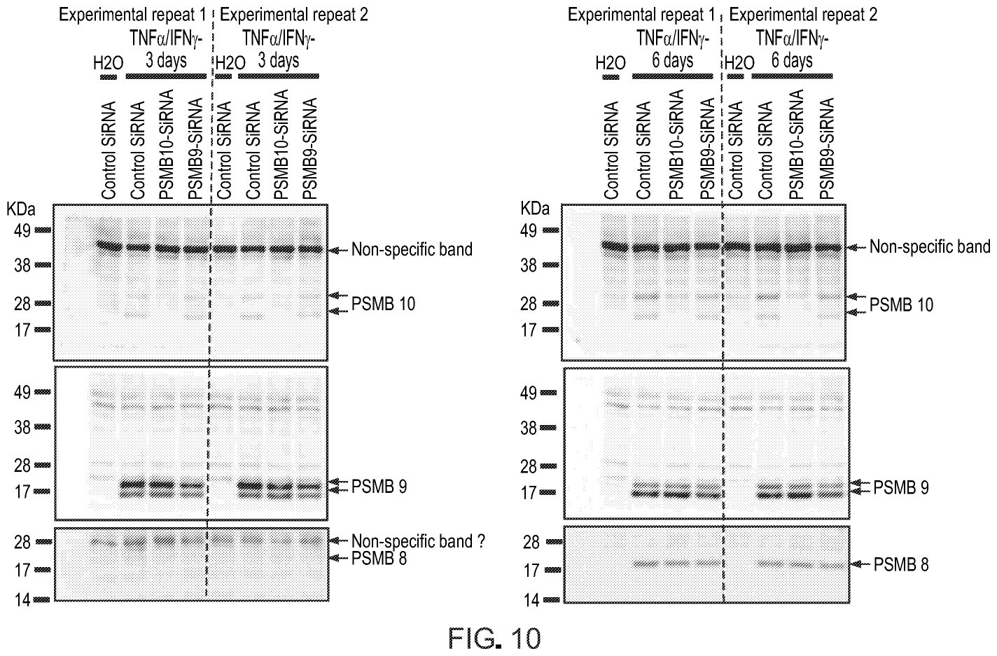

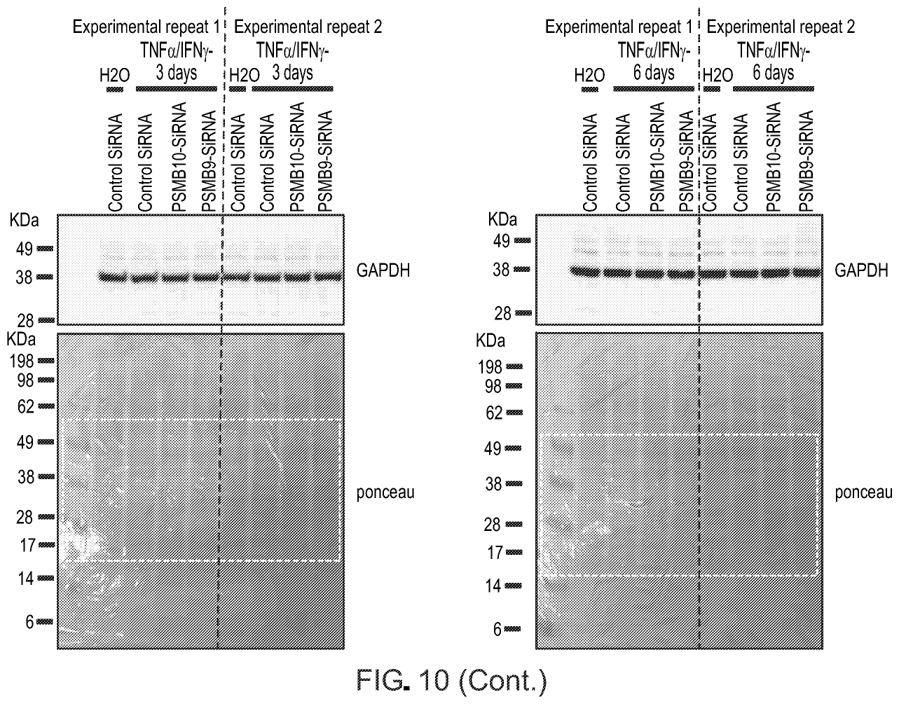

To further test that the IP can directly cleave Tau at sites consistent with SI material of AD patients (FIG. 1B), we incubated recombinant 0N4R and 2N4R Tau with purified IP. Overall, both isoforms exhibited very similar cleavage patterns and cleavage sites (FIG. 10). Multiple abundant cleavages were observed along the junction of R2 and R3 (N297-1308), including the high population frequency N-terminal cleavage sites observed in the tissue. C-terminal cleavages identified in the AD insoluble data at N368-K369i and D421-S422 were also detected after digestion of recombinant Tau with IP, supporting a potential role for the IP in generation of Tau fragments in AD.

Recombinant Tau Fragments 1a-4b Fibrilize and can Seed Aggregation In Vitro and in an HEK293 Tau-RD Biosensor Cell Line

Based on specificity to seed-competent tau, correlations with IP subunit abundance, and the cleavage sites observed in in vitro IP cleavage of tau, we selected a set of sites for evaluation of their potential to fibrillize and seed aggregation. This included 3 N-terminal cleavages immediately prior to the 306 VQIVYK311 hexapeptide, which is common between the 3R and 4R Tau isoforms and important for aggregation (see, e.g., Chen, D. et al. Tau local structure shields an amyloid-forming motif and controls aggregation propensity. Nat Commun 10, 2493, doi: 10.1038/s41467-019-10355-1 (2019)). While S305-V306 was not a prioritized cleavage site, it was included because of its critical position and detection in the in vitro cleavage of Tau by the IP. The two most prominent C-terminal sites were selected, N368-K369 and Y394-K395. We assign the following peptide nomenclature for clarity: N-terminal cleavages (1) N296-1297 (2) G303-G304, (3) G304-S305, (4) S305-V306 and C-terminal proteolytic cleavages (a) N368-K369 and (b) Y394-K395. When combined, the prioritized cleavage sites result in 8 unique Tau fragments 1a-4a and 1b-4b, which span the AD Tau filament core resolved by cryo-EM (FIG. 3B), although the a series lack the most C-terminal 10 residues

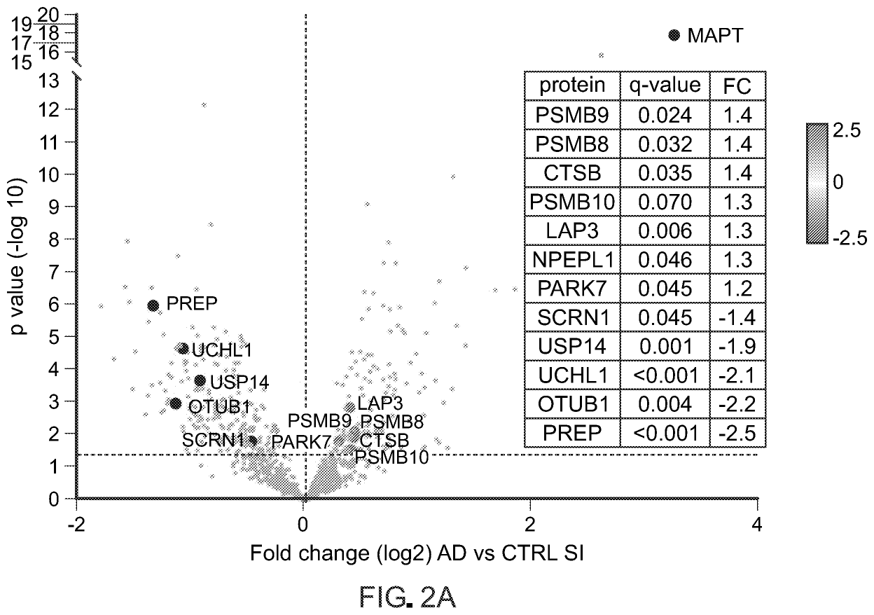

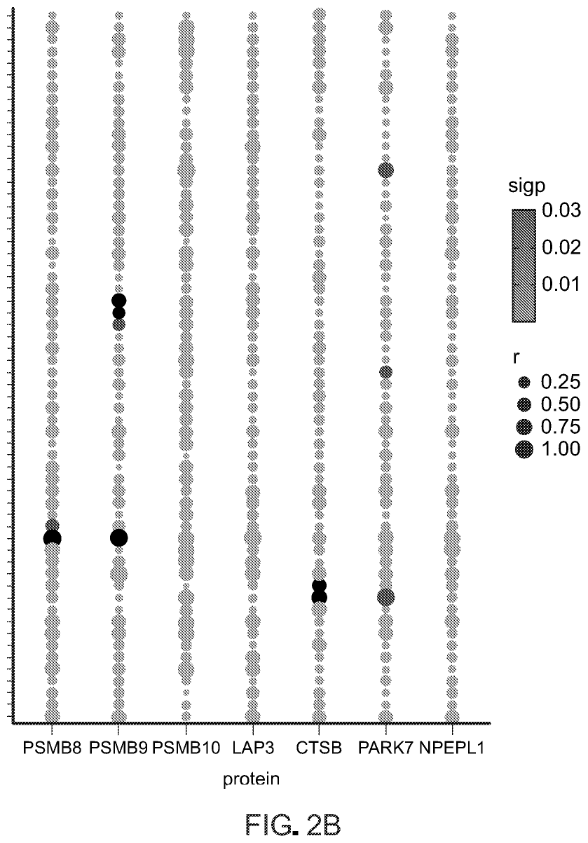

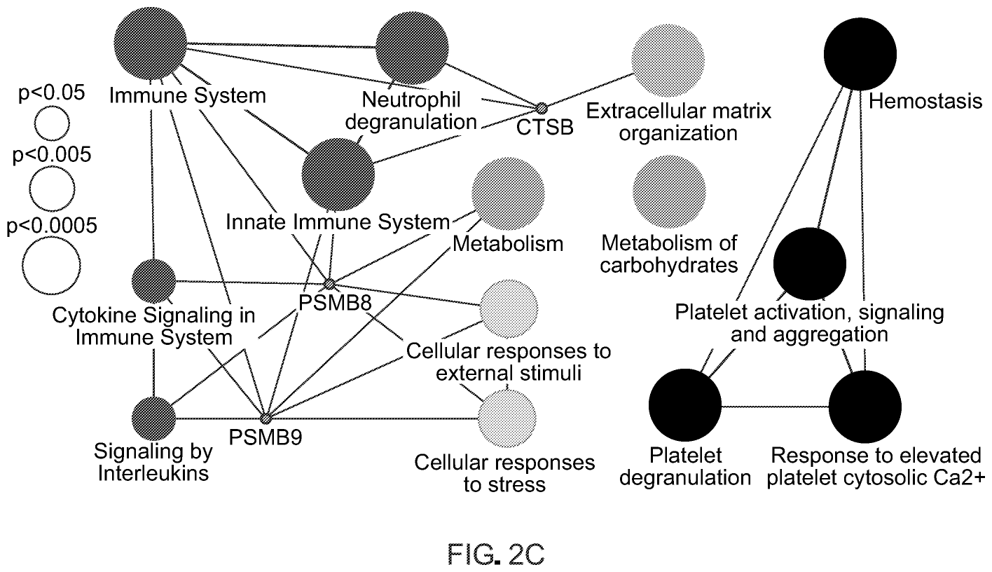

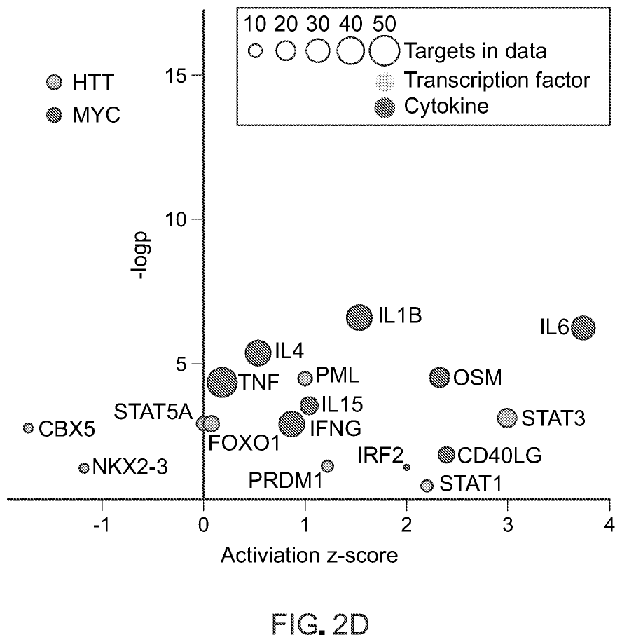

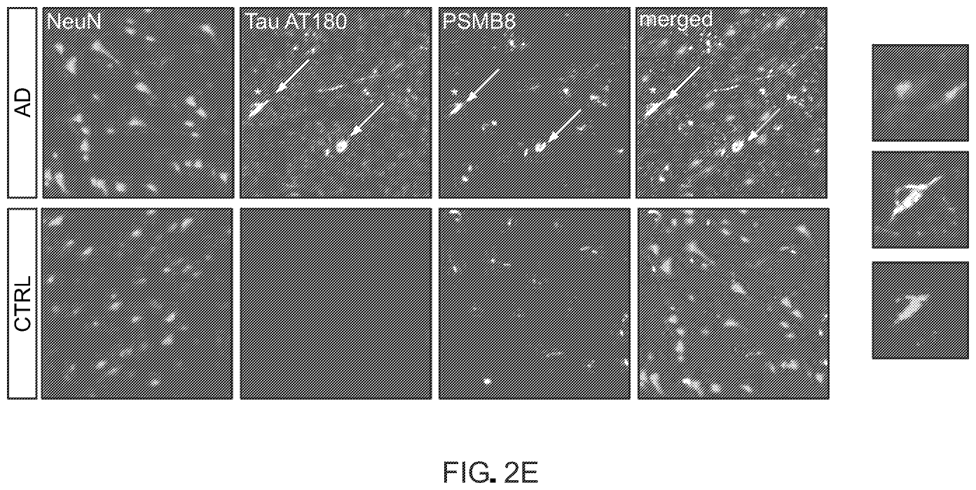

We generated recombinant peptides representing putative Tau fragments 1a-4b for detailed characterization of fibrillization and seeding properties. Fragments were first tested for their ability to form fibrils in vitro in the presence of heparin co-factor using the Thioflavin T (ThT) fluorescence assay. All fragments were found to be capable of forming fibrils, but the quantity of fibrils formed differed between the 8 peptides (FIG. 2A). The longer C-terminal b-series showed higher fibrillization propensity (p=0.0005), while extending the N-terminus had an insignificant effect (FIG. 2A). TEM with negative staining showed that all fibrils formed by these fragment peptides display characteristic paired helical filament-like structures.

Next we tested the ability of the fibrils generated from the fragments to seed aggregation in Tau-RD biosensor cells. The ranked seeding activity of peptide fibrils based on % of cells exhibiting distinct Tau puncta showed excellent agreement with that of the ThT assay: fibrils formed by b-peptides with the longer C-terminus were more effective at seeding, while the N-terminal sequence had a less pronounced effect (FIG. 2B);

We set out to test if the fibrillized Tau fragments were capable of seeding naive Tau without the addition of heparin, the most potent cofactor used to facilitate Tau aggregation. We used 2b as a representative peptide, given that 2b was the fragment most consistently observed across seeding-competent tissue preparations. A catalytic amount (1:40 molar ratio)) of 2b fibril was used as seed for a secondary fibrillization assay (FIG. 2C). Notably, this 2b-derived fibril seed was capable of inducing the formation of 2nd generation of fibrils. A control experiment using an equimolar effective concentration of heparin as was contained in the catalytic amount of 2b seed showed no detectable ThT fluorescence. Surprisingly, 2b fibril could seed 2nd generation fibrils of monomeric Tau 187. (FIGS. 2C and 2D). In stark contrast, seeds formed from Tau 187 failed to produce appreciable amounts of 2nd generation fibrils (FIGS. 2D and 2E). When normalized by the total mass of the seed or the cofactor, the results show that 2b seeds are much more effective at generating fibrils (FIG. 2D). The ThT data suggests that the C-terminal extension promotes the fibrillization of Tau and that formation of Tau fragments such as 2b can cause self-aggregation, as well as recruit longer, naïve, monomeric Tau in the seeding process.

Treatment of HEK293T Full-Length (FL) Tau Biosensor Cells with IFNγ/TNFα Results in IP-Dependent Tau Cleavage and Inclusion Formation

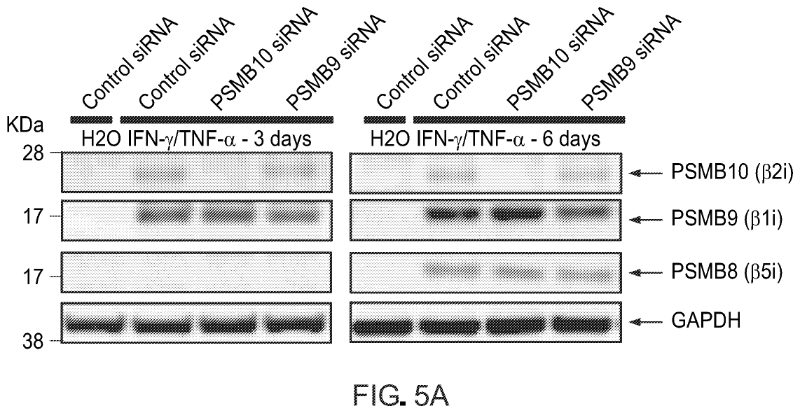

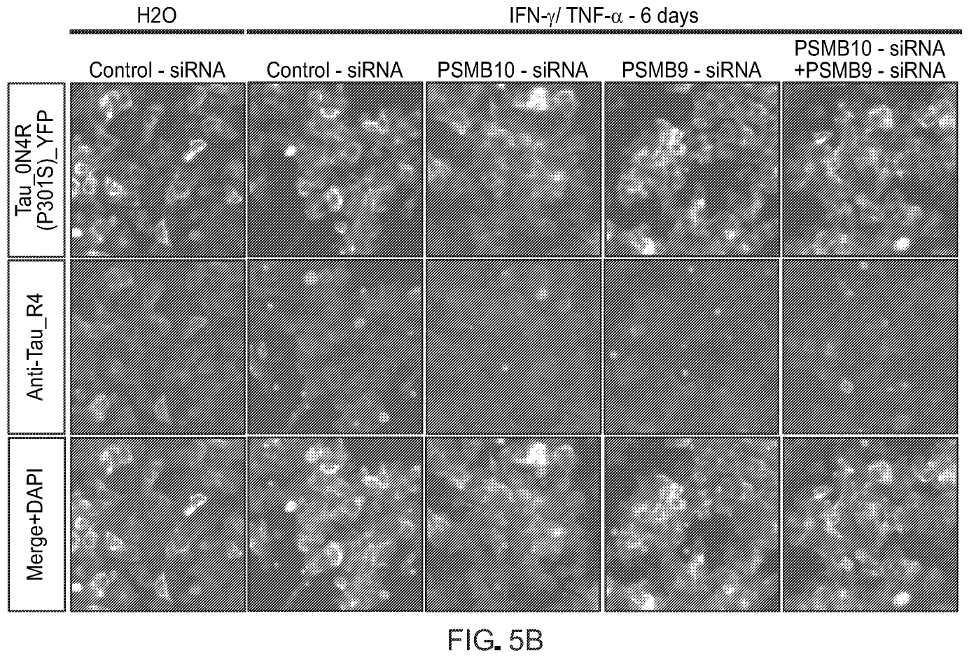

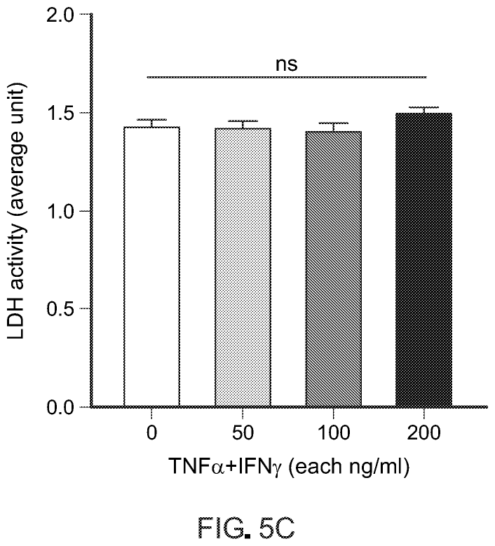

In order to model the effect of IP induction on Tau aggregation, we established a stable HEK2393T cell line expressing a FL human Tau isoform (0N4R), harboring an aggregation promoting P301S mutation with C-terminal fused EYFP. These cells were treated with a combination of IFN-γ and TNF-α to achieve robust immunoproteasome induction (see, e.g., Loukissa, A., Cardozo, C., Altschuller-Felberg, C. & Nelson, J. E. Control of LMP7 expression in human endothelial cells by cytokines regulating cellular and humoral immunity. Cytokine 12, 1326-1330, doi: 10.1006/cyto.2000.0717 (2000); and Shin, E.-C. et al. Virus-induced type I IFN stimulates generation of immunoproteasomes at the site of infection. Journal of Clinical Investigation 116, 3006-3014, doi: 10.1172/jci29832 (2006)). Cells were fixed the cells on days 3˜8 after treatment. Fluorescent imaging showed EYFP-fused Tau was expressed diffusely throughout the cytoplasm without accumulation or aggregation. Cytokine treatment had no significant effect on YFP expression pattern and there was no visible formation of YFP-positive inclusions. However, immunostaining with the anti Tau RD4 antibody, which binds an epitope in the R2 domain (see, e.g., de Silva, R. et al. Pathological inclusion bodies in tauopathies contain distinct complements of tau with three or four microtubule-binding repeat domains as demonstrated by new specific monoclonal antibodies. Neuropathol Appl Neurobiol 29, 288-302, doi: 10.1046/j. 1365-2990.2003.00463.x (2003)), showed immunoreactive inclusions (FIG. 5A). The numbers of inclusions increased over the course of treatment (FIG. 5A).

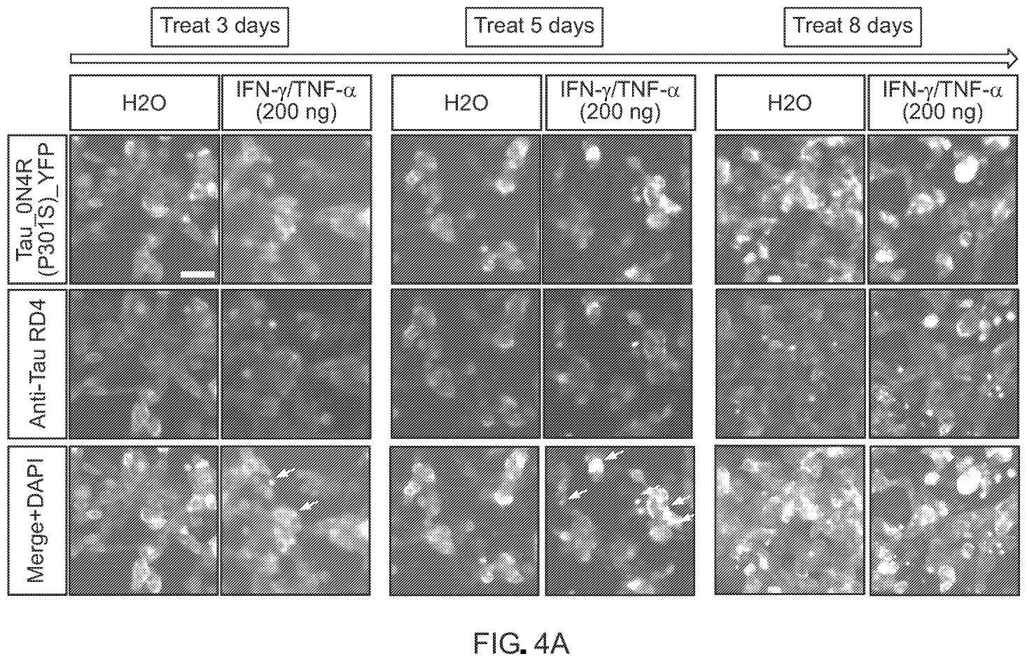

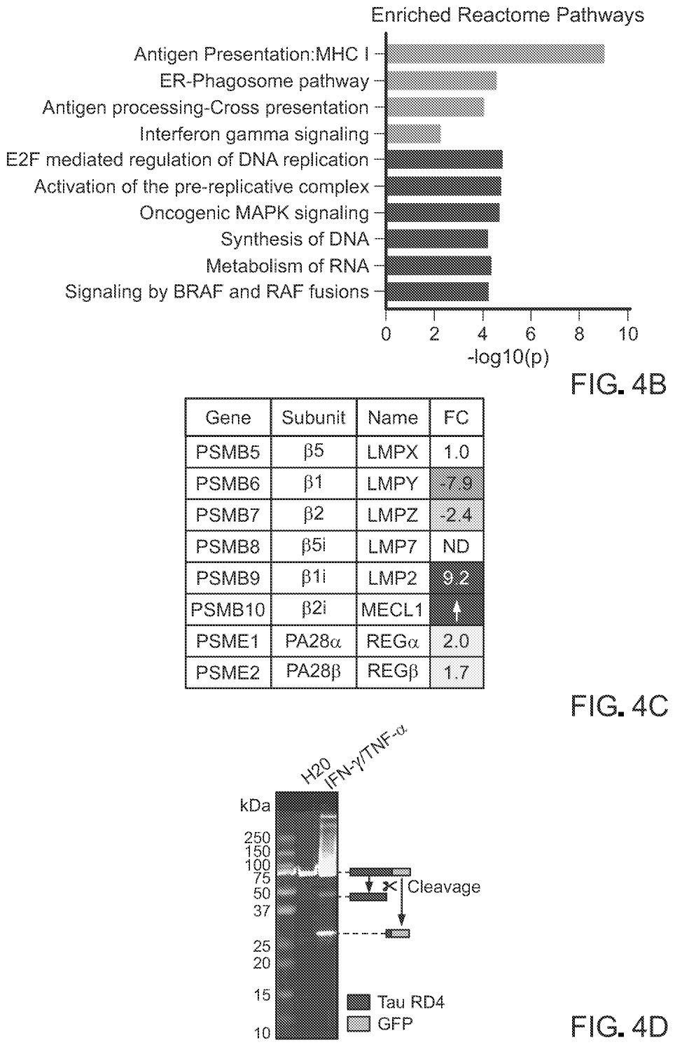

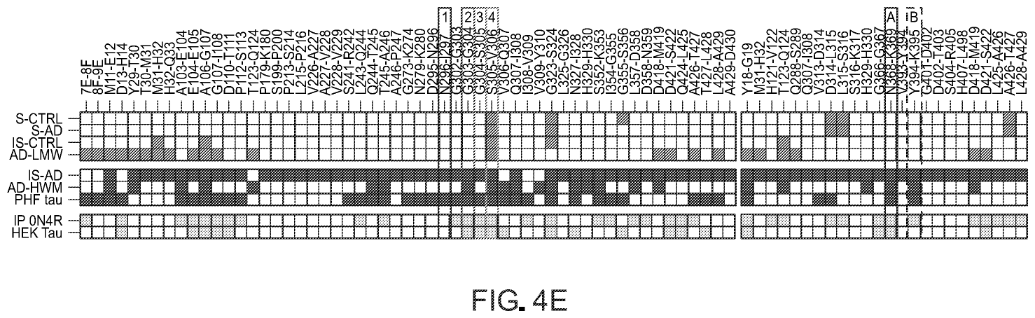

After 8 days cell lysates were processed for proteomic analysis to identify differentially expressed proteins. Pathway enrichment analysis showed activation of IFNγ signaling pathways. PSMB9 and 10 were upregulated by IFNγ-TNFα treatment in the proteomic data, and while PSMB8 was not detected by MS, it was increased by western blot (FIG. 5). The constitutive corresponding proteosome subunits i.e. PSMB 6 and 7 for PSMB9/10 were regulated in the opposite direction whereas no change in PSMB5 was observed. Components of the IFN γ induced 1 Is regulatory complex (PA28) PSME1 and PSME2 were also increased (FIG. 4C). Other proteases upregulated in AD SI fraction, including CTSB, LAP3, NPEPL1, and PARK7 were not increased by IFNγ/TNFα treatment indicating that Tau processing in HEK293T cells cannot be attributed to increased levels of these proteases.

The imaging data showing YFP negative, Tau 4R positive inclusions in response to cytokine treatment suggested cleavage of Tau between the RD4 Tau epitope and the YFP fluorophore. We blotted day 3 and 8 cell lysates for both YFP and 4R Tau. Western blotting clearly showed bands supporting C-terminal cleavage (FIG. 4). Given that the imaging and western blotting can only identify the C-terminal cleavage we performed proteomic analyses to comprehensively identify Tau cleavage sites. Certain cleavage sites were identified in the untreated condition, likely reflecting physiological degradation of Tau. Prioritized sites from AD postmortem tissue were identified in the HEK293T FL Tau cells after IFNγ/TNFα treatment (FIG. 4E), except sites 1 and b, supporting s the role of the IP in pathological Tau cleavage in human brain.

To prove the IFNγ/TNFα formation of Tau inclusions in HEK293T FL Tau biosensor cells was mediated by up-regulated IP subunits, we transfected the cells on the day before cytokine treatment with siRNA targeting PSMB 9 and 10. As expected, in positive controls the protein levels of PSMB 9 and 10 were dramatically increased 3 and 6 days after cytokine treatment. The siRNA efficiently and specifically knocked down the protein levels of PSMB9 and 10 in cytokine treated cells by approximately to 50% and 90%, respectively (FIG. 6A). The consistently observed IFNγ/TNFα induced EYFP-negative Tau inclusions were obviously reduced by application of PSMB10 and 9 siRNAs.

### Discussion

Tau aggregates characteristic of AD pathology consist of structurally modified and conformationally altered Tau proteoforms of unknown etiology. Accumulating genetic evidence points to neuroimmune components as crucial risk factors for AD, indicating neuroinflammation might be a primary driver in AD pathogenesis (ref). Our work identifies a mechanism of Tau processing by IPss to generate aggregation-prone fragments, directly linking neuroinflammation to initiation of Tau pathology in AD. Using our FLEXI-Tau approach complemented with conventional label-free proteomics, we identified cleavage throughout the Tau protein, and found a series of common AD-specific cleavage sites, including novel sites not reported previously. The extent and diversity of cleavage demonstrates substantial heterogeneity in composition of seed-competent tau species, despite the apparent homogeneity in cryoEM structures of AD-derived tau filaments².

Based on changes in abundance in the SI proteome and colocalization with neuronal pTau inclusions, the IP was implicated as a novel candidate protease for pathological tau cleavage. Increases in the IP have been described for Huntington's disease (see, e.g., Fernandez-Nogales, M. et al. Huntington's disease is a four-repeat tauopathy with tau nuclear rods. Nat Med 20, 881-885, doi: 10.1038/nm.3617 (2014)), DLB/PD (see, e.g., Ugras, S. et al. Induction of the Immunoproteasome Subunit Lmp7 Links Proteostasis and Immunity in alpha-Synuclein Aggregation Disorders. EBioMedicine 31, 307-319, doi: 10.1016/j.ebiom.2018.05.007 (2018)), and for AD at the transcriptome (see, e.g., Roy, E. R. et al. Type I interferon response drives neuroinflammation and synapse loss in Alzheimer disease. J Clin Invest 130, 1912-1930, doi: 10.1172/JCI133737 (2020)), protein, and activity levels. Additionally, in two recent AD GWAS metanalyses, PSMB9 was listed as a prioritized or potential causative gene (see, e.g., Jansen, I. E. et al. Genome-wide meta-analysis identifies new loci and functional pathways influencing Alzheimer's disease risk. Nat Genet 51, 404-413, doi: 10.1038/s41588-018-0311-9 (2019); and Kunkle, B. W. et al. Genetic meta-analysis of diagnosed Alzheimer's disease identifies new risk loci and implicates Abeta, tau, immunity and lipid processing. Nat Genet 51, 414-430, doi: 10.1038/s41588-019-0358-2 (2019)). However, previous studies on the IP in AD have focused on glial cell expression and have not examined direct effects on Tau. IP subunits are induced by proinflammatory cytokines such as TNFα/IFNγ, and replace the catalytic subunits of the constitutive 20S proteasomes to resolve proteotoxic stress and produce peptides for antigen presentation. We found evidence for activation of cell stress and immune pathways, and proinflammatory cytokine signaling in the SI fraction, explaining IP induction. We show here that the IP can cleave recombinant FL Tau at AD-specific cleavage sites surrounding the protofilament core at sites that have not been reported for the constitutive proteasome in similar experiments (see, e.g., Ukmar-Godec, T. et al. Proteasomal degradation of the intrinsically disordered protein tau at single-residue resolution. Sci Adv 6, eaba3916, doi: 10.1126/sciadv.aba3916 (2020)). Furthermore, abundance of the IP subunits correlated with intensities of cleavages surrounding the AD protofilament, prompting us to investigate the potential role of these cleavages in Tau aggregation.

Out of the prioritized cleavage sites, 1 and 2 were novel, and while cleavage at site 3 and b have both been described, the properties of the fragments or the responsible proteases have not been determined (see, e.g., Derisbourg, M. et al. Role of the Tau N-terminal region in microtubule stabilization revealed by newendogenous truncated forms. Scientific Reports 5, doi: 10.1038/srep09659 (2015)). The N-terminal cleavage sites 1-3 are all located immediately prior to the start of the R3 hexapeptide (VQIVYK), which was shown to be essential for in vitro fibrillization and seeding in cells (see, e.g., Stohr, J. et al. A 31-residue peptide induces aggregation of tau's microtubule-binding region in cells. Nat Chem 9, 874-881, doi: 10.1038/nchem.2754 (2017); and Von Bergen, M. et al. Assembly of tau protein into Alzheimer paired helical filaments depends on a local sequence motif ((306) VQIVYK (311)) forming beta structure. Proc Natl Acad Sci USA 97, 5129-5134, doi: 10.1073/pnas.97.10.5129 (2000).

We hypothesize that endogenous cleavage sites 1, 2 & 3 would expose the hexapeptide, accounting for their ability to fibrillize and seed aggregation in vitro. While all peptides could fibrillize, peptides with C-terminal cleavage at b had higher activity than the peptides ending at a, suggesting the extended C-terminus aids in fibril formation. This is particularly interesting because C-terminal cleavage at site a (N368-K368) has previously been proposed to generate aggregation-prone fragments (see, e.g., Zhang, Z. et al. Cleavage of tau by asparagine endopeptidase mediates the neurofibrillary pathology in Alzheimer's disease. Nat Med 20, 1254-1262, doi: 10.1038/nm.3700 (2014)).

Importantly, we show for one representative peptide (2b) that it can initiate fibrillization of larger Tau species in a 2nd generation seeding experiment. Supporting this observation are studies of the fragment 297-391 originally identified as a component of PHFs (see, e.g., Novak, M., Kabat, J. & Wischik, C. M. Molecular characterization of the minimal protease resistant tau unit of the Alzheimer's disease paired helical filament. EMBO J 12, 365-370 (1993)) which closely resembles our b fragments in length and sequence. This fragment was shown to form fibrils the absence of heparin resembling AD PHFs on a macromolecular scale (see, e.g., Al-Hilaly, Y. K. et al. Alzheimer's Disease-like Paired Helical Filament Assembly from Truncated Tau Protein Is Independent of Disulfide Crosslinking. J Mol Biol 429, 3650-3665, doi: 10.1016/j.jmb.2017.09.007 (2017); and Al-Hilaly, Y. K. et al. Tau (297-391) forms filaments that structurally mimic the core of paired helical filaments in Alzheimer's disease brain. FEBS Lett 594, 944-950, doi: 10.1002/1873-3468.13675 (2020). Therefore, fragments produced by IP-cleavage of Tau have the necessary properties to initiate aggregation.

Modeling the increased IP expression observed in the proteomics data by treating FL Tau-expressing biosensor cells with TNFα/IFNγ resulted in Tau cleavage at AD-specific sites, and spontaneous formation of Tau inclusions in the absence of seeds. While we cannot exclude contributions of other proteases, the reduction in inclusion formation by IP siRNA knockdown demonstrates an important role for the IP in this process.

Together these data form a compelling case for a role of IP-cleaved Tau species in the initiation of pathological Tau aggregation. We propose that cell-intrinsic stress responses and inflammatory cytokines originating from local secretion, glia, or systematic inflammation, increase neuronal IP expression, linking known AD risk factors to Tau processing. Many genetic risk genes for AD are expressed primarily in microglia, one of the potential sources of proinflammatory cues^{32, 33}. Moreover, AD modifiable risk factors (see, e.g., Livingston, G. et al. Dementia prevention, intervention, and care: 2020 report of the Lancet Commission. The Lancet 396, 413-446, doi: 10.1016/s0140-6736 (20) 30367-6 (2020)) such as smoking, diabetes, TBI, obesity are associated with increased systemic and neuro inflammation. Aside from extrinsic proinflammatory cytokines, oxidative stress, exposure to protein aggregates, and proposed viral/bacterial risk factors could all trigger IP expression in neurons. Neuronal exposure to amyloid beta increases IP expression, which could in part mediate the observed synergy between amyloid beta and Tau pathology (see, e.g., Busche, M. A. & Hyman, B. T. Synergy between amyloid-beta and tau in Alzheimer's disease. Nat Neurosci, doi: 10.1038/s41593-020-0687-6 (2020)). However, Tau cleavage is one of many mechanisms by which inflammation can affect AD pathology. Previous functional studies have shown that inflammation-induced Tau phosphorylation can play a role in driving pathology in a tauopathy mouse model (see, e.g., Bhaskar, K. et al. Regulation of tau pathology by the microglial fractalkine receptor. Neuron 68, 19-31, doi: 10.1016/j.neuron.2010.08.023 (2010)). Furthermore, whether increasing IP expression is sufficient to trigger pathological proteolytic cleavage of Tau, or if Tau is targeted as a consequence of PTMs remains to be determined. Tau is extensively modified (Wesseling et al 2020) in ways that affect proteolytic cleavage, and PTMs can affect fibrillization kinetics, resulting in complex interactions that need to be characterized in future studies.

### CONCLUSION

Understanding the vast diversity of Tau proteoforms, and how this landscape is altered by the cellular environment under disease conditions is key to the development of effective therapeutics for AD and other Tauopathies. Our characterization of Tau cleavage in a large cohort of AD patients combined with functional analysis provides comprehensive insight into the disease and establishes a convincing model where inflammatory cytokines and cellular stress result in generation of Tau seeds. This model represents an early step in the initiation of Tau pathology and provides multiple potential treatment opportunities, such as modulation of neuroinflammation, cleaved Tau neoepitope antibodies, and IP inhibition. Indeed, inhibitors for PSMB9/6 have demonstrated efficacy in AD mouse models, although not directly in models of Tauopathy (see, e.g., Yeo, I. J. et al. A dual inhibitor of the proteasome catalytic subunits LMP2 and Y attenuates disease progression in mouse models of Alzheimer's disease. Sci Rep 9, 18393, doi: 10.1038/s41598-019-54846-z (2019)). Lastly, the data presented in this manuscript has relevance for ongoing Tau antibody development projects, as it emphasizes the importance of epitope selection in light of the extensive cleavage observed in seed-competent Tau.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a tauopathy in a subject in need thereof, the method comprising:
(a) identifying the subject having a tauopathy;
(b) administering to the subject an effective amount of a pharmaceutical composition comprising an agent that decreases the expression and/or activity of immunoproteasome (IP),
thereby treating the tauopathy in the subject.

2. The method of claim 1, wherein the subject has Alzheimer's Disease (AD).

3. The method of claim 1, wherein the agent decreases the expression and/or activity of an immunoproteasome-related protein whose level is upregulated in a subject having tauopathy.

4. The method of claim 1, wherein the immunoproteasome-related protein is selected from the group consisting of PSMB 8, PSMB 9, PSMB 10, mitochondrial inner membrane protease, serine protease HTRA1, serine protease HTRA2, inactive Ufm1-specific protease 1, calpain small subunit 1, thimet oligopeptidase, puromycin-sensitive aminopeptidase, signal peptidase complex subunit SEC11a, endoplasmic reticulum aminopeptidase 1, caspase 1, lysomal pro-X carboxypeptidase, peptidase inhibitor 16, prolyl endopeptidase, cytosol aminopeptidase, isoaspartyl peptidase, Xaa-pro dipeptidase, dipeptidyl aminopeptidase-like protein 16, isoaspartyl peptidase, cytosol aminopeptidase, probable aminopeptidase NPEPL1, prolyl endopeptidase, carboxypeptidase Q, puromycin-sensitive aminoprptidase, and dipeptidyl peptidase 3.

5. The method of claim 1, wherein the agent increases the expression and/or activity of an immunoproteasome-related protein whose level is downregulated in a subject having tauopathy.

6. The method of claim 5, wherein the agent increases the expression and/or activity of deubiquitinase.

7. The method of claim 1, wherein the agent decreases the expression and/or activity of a cytokine or a cytokine receptor, wherein the cytokine is selected from the group consisting of IL-6, OSM (IL6 family), IL-1B, IL-15, IFN-γ, and TNF-α.

8. (canceled)

9. The method of claim 1, wherein the agent decreases the expression and/or activity of a transcription factor, wherein the transcription factor is selected from the group consisting of STAT1/3, SMARCA4, and IRF2.

10. (canceled)

11. The method of claim 1, wherein the agent is a small molecule, a peptide, a stapled peptide, an siRNA, an antisense oligonucleotide (ASO), or an antibody.

12. The method of claim 1, wherein the agent reduces aggregation of tau peptides, the seeding competence of tau peptides, or fibrillization of tau peptides in the subject.

13. (canceled)

14. (canceled)

15. A method for screening a therapeutic agent for treating a tauopathy, the method comprising:
(a) providing a plurality of cells that express tau peptides;
(b) subjecting the plurality of cells to one or more stress signals, wherein the stress signals induce the aggregation of tau peptides;
(c) contacting the plurality of cells from (b) with a candidate therapeutic agent;
(d) comparing the levels of tau peptide aggregation before and after the contacting of the candidate therapeutic agent;
(e) selecting the therapeutic agent if the aggregation of tau peptides is decreased. thereby screening the therapeutic agent.

16. The method of claim 15, further comprising measuring aggregation of tau peptide prior to (d).

17. The method of claim 15, further comprising identifying fragments of tau peptides that comprises one or more cleavage sites.

18. (canceled)

19. The method of claim 15, wherein the therapeutic agent targets one or more cleavage sites on tau peptide, or one or more of post-translational modifications (PTMs) on tau peptide.

20. The method of claim 19, wherein the therapeutic agent is an antibody.

21. (canceled)

22. The method of claim 15, wherein the tauopathy is Alzheimer's Disease (AD).

23. The method of claim 15, wherein the therapeutic agent decreases the expression and/or activity of an immunoproteasome-related protein whose level is upregulated in a subject having tauopathy or increases the expression and/or activity of an immunoproteasome-related protein whose level is downregulated in a subject having tauopathy.

24. (canceled)

25. The method of claim 15, wherein the therapeutic agent decreases the expression and/or activity of a cytokine or a cytokine receptor, or a transcription factor.

26. (canceled)

27. The method of claim 15, wherein the therapeutic agent is a small molecule, a peptide, a stapled peptide, an siRNA, an antisense oligonucleotide (ASO), or an antibody.

28. The method of claim 15, wherein the therapeutic agent reduces aggregation of tau peptides, the seeding competence of tau peptides or fibrillization of tau peptides in the subject.

29. (canceled)

30. (canceled)
